(19) United States
(12) Patent Application Publication
(10) Pub. No.: US 2026/0295045 A1
(43) Pub. Date: Oct. 1, 2026

(54) COMPOSITIONS COMPRISING WATER AND ULTRAFINE BUBBLES AND METHODS OF USING THEREOF IN INDUSTRIAL AND MANUFACTURING PROCESSES

(71) Applicant: Hydrosome IP, LLC, Westmont, IL (US)

(72) Inventors: Carly S. Hanson, Naperville, IL (US); Jay Chamberlain, Burr Ridge, IL (US); Cassandra Villanueva, Chicago, IL (US); John Nicholas Jackowetz, Rochester, NY (US)

(21) Appl. No.: 19/573,774

(22) Filed: Mar. 20, 2026

Related U.S. Application Data
(60) Provisional application No. 63/775,614, filed on Mar. 21, 2025.

(51) Int. Cl. A61K 41/00; A61K 33/00; A61P 1/14; A61P 29/00; C02F 1/34

(52) U.S. Cl. CPC A61K 41/0028; A61K 33/00; A61P 1/14; A61P 29/00; C02F 1/34

(57) ABSTRACT

The disclosure provides compositions that comprise water and ultrafine bubbles, which compositions have improved cell permeability, bioavailability and stability. The present disclosure also relates to methods of using ultrafine bubble water compositions in various applications and methods, including but not limited to enhancement of foam stabilization and emulsion stability, modulation of gut microbiota composition, which may be useful in a variety of applications including application in non-invasive microbiome therapeutics, and inhibition of oxidation of ascorbic acid for use in cosmetic and other applications.

### FIELD

The present disclosure generally relates to compositions that comprise water and ultrafine bubbles, which compositions have improved cell permeability, bioavailability and stability. The present disclosure also relates to methods of using ultrafine bubble water compositions in various applications and methods, including but not limited to, improving spray drying efficiency and product quality by incorporating ultrafine bubbles into oil-in-water emulsions prior to atomization and drying, enhancement of foam stabilization and emulsion stability, modulation of gut microbiota composition, and inhibition of oxidation of ascorbic acid for use in cosmetic and other applications.

### BACKGROUND

Ultrafine bubbles in aqueous compositions can be generated when “cavitation” occurs. There are two fundamental types of cavitation: (1) vaporous or “hard” cavitation and (2) gaseous or “soft” cavitation. Vaporous cavitation occurs when the pressure in a liquid drops below the vapor pressure of the liquid, resulting in the formation of unstable low-pressure voids or bubbles formed from vaporized particles or molecules of the liquid itself. By contrast, gaseous cavitation occurs when gases dissolved within a liquid fall out of solution with decreasing pressure, typically at pressures higher than the vapor pressure of the liquid itself-creating bubbles formed from particles or molecules of the liquid and the released gases.

Typical aqueous ultrafine bubbles are created as a result of vaporous or “hard” cavitation, when the resulting voids collapse and form shockwaves. The shockwaves facilitate the formation of ultrafine bubbles from exogenous non-dissolved gases. These ultrafine bubbles have substantially different structural, functional, and stability characteristics than ultrafine bubbles formed from “gaseous” cavitation. In aqueous compositions, ultrafine bubbles formed from “hard” cavitation (which comprise bubbles including water molecules surrounding exogenously provided gases) have substantially different physical characteristics in terms of structure, function, and stability than ultrafine bubbles formed from “soft” cavitation (which comprise bubbles including water molecules surrounding gases released from solution within the water).

With respect to aqueous compositions, it is well known that the organization of water molecules influences the stability, solubility, and bioavailability of any solutes dissolved within. As the organization of water molecules in compositions having ultrafine bubbles produced via vaporous or “hard” cavitation differs substantially from that of the organization of water molecules in compositions containing ultrafine bubbles produced via gaseous or “soft” cavitation, the stability, solubility, and bioavailability of solutes dissolved within the two compositions may be substantially different. It would be beneficial to produce aqueous compositions including water and ultrafine bubbles comprising gases released from solution in water—that is, produced via gaseous or “soft” cavitation—which compositions have improved stability, solubility, and bioavailability as compared to compositions including no ultrafine bubbles or solutions comprising or consisting of aqueous ultrafine bubbles formed via vaporous or “hard” cavitation. There also exists a need for compositions that comprise water, ultrafine bubbles comprising gases released from solution in water, and a non-gaseous solute that have improved bioavailability, solubility, and/or stability.

Ultrafine bubble-infused water significantly enhances foam stability in oil-water emulsions and beverage systems through interfacial stabilization mechanisms. Ultrafine bubbles position at oil-water interfaces, acting as nano-scale stabilizers that prevent droplet coalescence and maintain foam structure. Gas type selection in ultrafine bubble water enables targeted control of foam characteristics. Nitrogen-filled ultrafine bubbles optimize foam longevity and texture in alcoholic beverages. Air-filled ultrafine bubbles provide enhanced foam volume in sweetener-based systems. This gas-selectivity allows formulation optimization for specific product requirements without altering base ingredients. Thus, there is a need for stabilization of foam having applications in cosmetic emulsions, food emulsions, beverage foaming and functional foods requiring stable foams.

Recent advances in ultrafine bubble (UFB) generation technology have led to an increased interest in their biological applications, particularly in the realm of gut health. This increased attention coincides with increased consumer awareness regarding the gut microbiome's role in overall health. The gut microbiome, comprising trillions of microorganisms living in our digestive tract, plays a crucial role in everything from immune function and metabolism to mental health and disease prevention. Research has shown that the diversity and balance of these microbial communities can significantly impact overall health outcomes, with imbalances being linked to conditions ranging from inflammatory bowel disease and obesity to anxiety and depression. The gut-brain axis, mediated through microbiome derived metabolites and immune system interactions, has emerged as a key pathway through which gut bacteria influence systemic health. In particular, Firmicutes and Bacteroidetes represent the two dominant bacterial phyla in a healthy human gut, typically comprising 80-90% of the microbial community. The Firmicutes-to-Bacteroidetes (F/B) ratio serves as a key indicator of gut health, with elevated ratios historically associated with obesity and metabolic dysfunction, though recent evidence suggests this relationship is more complex than initially proposed.

Among the crucial metabolites produced by gut bacteria, short-chain fatty acids (SCFAs) have garnered particular attention as they serve as an energy source for gut bacteria and intestinal cells and regulate gut barrier integrity. High SCFA production is associated with positive health outcomes, including increased beneficial bacterial growth and reduced pathogenic bacteria linked to several diseases and health deficiencies. The most abundant SCFAs-acetate, propionate, and butyrate-play distinct roles in maintaining gut health, with butyrate particularly important as the primary energy source for colonocytes. Additionally, inflammatory markers such as cytokines are of interest, as elevated cytokine levels are linked with inflammatory and autoimmune diseases, including irritable bowel disease (IBD), arthritis, cardiovascular disease, and diabetes. The balance between pro-inflammatory and anti-inflammatory cytokines is crucial for maintaining immune homeostasis and gut barrier function.

Thus, a need exists for modulation of gut microbiota compositions via ultrafine bubble water. Utilizing ultrafine bubble compositions produced according to the present disclosure and used to modulate gut microbiota may result in non-invasive microbiome therapeutics, including applications in IBD, metabolic syndrome and obesity management.

A need also exists for modulation of gut microbiota compositions, resulting the enhancement of short chain fatty acid (SCFA) production. Utilizing ultrafine bubble compositions produced according to the present disclosure to enhance SCFA production may be advantageous to typical SCFA supplementation currently available.

A need also exists for modulation of gut microbiota compositions, resulting in a reduction of inflammatory markers. Utilizing ultrafine bubble compositions produced according to the present disclosure may be useful as a non-drug approach to inflammation management, including applications in IBD, arthritis, metabolic inflammation and general wellness.

Ultrafine bubbles have also been shown to inhibit the autoxidation of ascorbic acid in aqueous solutions. This inhibitory effect is observed for both Air-UFBs and N₂-UFBs and inhibition does not correlate to type of gas entrained. This is unique from previous reports utilizing H₂-UFBs or specifically utilizing ultra-small UFBs (<10 nm) as antioxidants. The inhibition of ascorbic acid (Vitamin C) oxidation the use of other shelf-life extension chemicals such as sodium ascorbate, ascorbyl glucoside, glutathione, ergothioneine, niacinamide, riboflavin (Vitamin B2), alpha-lipoic acid, quercetin (as glycosides), rutin, ferulic acid, caffeic acid, chlorogenic acid, gallic acid, epigallocatechin gallate (EGCG), ellagic acid, rosmarinic acid, sodium erythorbate, erythorbic acid, propyl gallate, octyl gallate, dodecyl gallate, sodium sulfite, sodium bisulfite, sodium metabisulfite, potassium metabisulfite, taurine, carnosine, anserine, histidine, cysteine, N-acetylcysteine, methionine, pyrroloquinoline quinone (PQQ), melatonin, uric acid, lactoferrin, albumin, superoxide dismutase (SOD), catalase, polydatin, fisetin, kaempferol (as glycosides), luteolin (as glycosides), hesperidin, naringin, diosmin, baicalin, scutellarin, phlorizin, hydroxytyrosol, oleuropein, tyrosol, protocatechuic acid, syringic acid, homogentisic acid, procyanidins, proanthocyanidins, ellagitannins, gallotannins, punicalin, punicalagin, kojic acid, azelaic acid, tranexamic acid, arbutin, deoxyarbutin, thioctic acid, phytic acid (inositol hexaphosphate), trehalose, anthocyanins, betanin is advantageous for cosmetic applications, where most formulations opt for more stable, albeit less effect derivatives of Vitamin C. This application could also be extended to beverages or food products that include Vitamin C as an added nutrient to potentially extend their shelf lives. Thus, there is a need to stabilize and/or inhibit the oxidation of ascorbic acid utilizing ultrafine bubbles.

Emulsions are fundamental to countless consumer products, from mayonnaise and salad dressings to cosmetic creams and lotions. These complex systems consist of two immiscible phases—typically oil and water—that require precise formulation to maintain stability. Conventional approaches to improving emulsion stability often rely on: Increasing emulsifier concentrations; adding synthetic stabilizers; modifying processing conditions; incorporating texture-enhancing additives. However, these conventional approaches often result in complex ingredient profiles, increased manufacturing costs, and more intricate production processes. As consumer preference shifts toward clean-label products with simplified formulations, manufacturers face the challenge of maintaining or improving product performance while reducing additive dependency. Therefore, a need exists for improving emulsion and foam stability and texture in cosmetic and food formulations without synthetic additives through utilization of ultrafine bubble technology.

Emulsions further are fundamental to other applications, including spray drying and forming spray-dried powders. Typically, spray drying technologies incorporate a material to be spray dried into a liquid, typically water to form a feed material, atomizing the feed material, and drying the same to form powder particles. Such a process is typically utilized across food, pharmaceutical, and cosmetic industries for encapsulation applications. A need, therefore, exists for improving emulsions and methods of spray drying that improves emulsion particle sizes, particle counts, eliminates particle size collapse through the use of ultrafine bubbles.

### SUMMARY

The present disclosure generally relates to compositions comprising water and ultrafine bubbles. The present disclosure also relates to methods of using ultrafine bubble water compositions in various manufacturing and industrial applications and methods, including but not limited to enhancement of foam stabilization and emulsion stability for use in cosmetics and food industry; modulation of gut microbiota composition for use in non-invasive microbiome therapeutics, nutritional supplementation and anti-inflammatory therapeutics; and, inhibition of oxidation of ascorbic acid for use in cosmetics and other applications.

Ultrafine bubbles (UFB) may comprise water and gases released from solution in water. In some embodiments, the ultrafine bubbles are formed from gases released from solution in water. In some embodiments, the gases released from solution in water to form the ultrafine bubbles are released via gaseous cavitation. In some embodiments, the gases are released as a result of gaseous cavitation. In some embodiments, the gases are released when the water in the composition is subjected to a combination of hydrodynamic cavitation, shear forces, and low-pressure/room temperature boiling to form the ultrafine bubbles.

In another aspect, a method for producing a composition comprising water and ultrafine bubbles is provided. The method includes generating and adding microbubbles to a source of water. Then, the water with added microbubbles is subjected to one or more of hydrodynamic cavitation, shear forces, and low pressure/room temperature boiling to produce ultrafine bubbles formed by release of dissolved gases from the water and/or the microbubbles. The resulting composition has formed ultrafine bubbles at a concentration of up to 10¹² ultrafine bubbles/mL.

In another aspect, a method designed to preserve ultrafine bubbles indefinitely within a solution is provided. The formulation consists of an ultrafine bubble suspension combined with an ingredient that increases the viscosity of the final product. By enhancing the viscosity, the mobility of the ultrafine bubbles is restricted, effectively preventing them from bursting or coalescing. This stabilization mechanism ensures that the ultrafine bubbles remain uniformly dispersed within the solution over extended periods, making the formulation suitable for various applications, including skincare, pharmaceutical, and consumable products, as well as application in manufacturing processes, including fermentation.

A method for enhancing foam stability in a beverage is provided. The method comprises providing a beverage base, adding water comprising ultrafine bubbles filled with nitrogen gas, the ultrafine bubbles having a mean diameter of 50-500 nm at a concentration of 1.0×10⁶ to 1.0×10¹² bubbles/mL, wherein the resulting beverage exhibits enhanced foam retention time compared to a control beverage made with water lacking ultrafine bubbles.

In another aspect, a method for selecting gas composition in ultrafine bubble water for foam applications is provided. The method for alcoholic beverage applications comprises using nitrogen-filled ultrafine bubbles, and for non-alcoholic sweetener-containing applications, the method comprises using air-filled ultrafine bubbles, wherein said ultrafine bubbles have a mean diameter of 50-500 nm at a concentration of 1.0×10⁶ to 1.0×10¹² bubbles/mL.

In a further aspect, a method of modulating gut microbiota composition in a gut of a mammal, is provided. The method comprises the steps of providing a drinking water composition comprising ultrafine bubbles formed via gaseous cavitation for ingestion by the mammal, wherein when the drinking water composition comprising the ultrafine bubbles is ingested by the mammal for modulating microbiota populations within the gut of the mammal.

In yet another aspect, a method of using ultrafine bubbles for modulation of gut microbiota compositions is provided. The method of modulating gut microbiota composition in a mammal comprises providing UFB-enriched drinking water formed via gaseous cavitation at 1.7×10⁶ to 1.0×10¹² UFBs/mL, wherein ingestion of the UFB-enriched drinking water results in increased Bacteroidetes and/or decreased Firmicutes populations and reduced F/B ratio.

In yet another aspect, a method of using ultrafine bubbles for modulation of gut microbiota compositions, resulting the enhancement of short chain fatty acid (SCFA) production is provided. The method of enhancing short chain fatty acid production comprises providing drinking water with air-filled UFBs at 1.0×10⁶ to 1.0×10¹² UFBs/mL, wherein ingestion of the UFB-enriched drinking water increases butyrate by at least 50%, valerate by at least 60%, with acetate unchanged. Utilizing ultrafine bubble compositions produced according to the present disclosure to enhance SCFA production may be advantageous over typical SCFA supplementation currently available.

In yet a further aspect, a method of using ultrafine bubbles for modulation of gut microbiota compositions, resulting in a reduction of inflammatory markers is provided. The method of reducing inflammatory markers via microbiome modulation comprises providing UFB-enriched drinking water for at least 6 weeks, wherein ingestion of the UFB-enriched drinking water reduces pro-inflammatory cytokines (TNF-α, IL-1β levels) by at least 80%, reduces IL-1β levels by at least 40%, reduces regulatory cytokines (IL-10) levels by at least 65%, wherein the reductions being associated with changes in gut microbiota composition.

Utilizing ultrafine bubble compositions produced according to the present disclosure may be useful as a non-drug approach to inflammation management, including applications in IBD, arthritis, metabolic inflammation and general wellness.

In yet another aspect, a method of using ultrafine bubbles for modulation of gut microbiota compositions for therapeutic applications of microbiome-related conditions is provided. Utilizing ultrafine bubble compositions produced according to the present disclosure may be useful as non-invasive therapeutic intervention for conditions requiring microbiome modulation, involving enhanced SCFA production and reduced inflammatory markers through endogenous microbiome shifts.

In another aspect, a method of inhibiting oxidation of ascorbic acid in an aqueous solution is provided. The method comprises incorporating ultrafine bubbles (UFBs) having a mean diameter of less than 1000 nm into the aqueous solution, wherein the UFBs are generated by hydrodynamic cavitation and the rate of ascorbic acid autoxidation is reduced relative to a comparable solution without UFBs, and wherein the inhibitory effect is independent of the gas species entrained within the UFBs.

In yet another aspect, an aqueous composition is provided. The compositions comprises ascorbic acid (Vitamin C); and ultrafine bubbles having a mean diameter of less than 1000 nm and a concentration of at least 1×10⁸ particles/mL, wherein the rate of oxidative degradation of ascorbic acid is reduced relative to an equivalent composition without ultrafine bubbles.

A method of inhibiting oxidation of ascorbic acid in an aqueous solution is provided. The method comprises incorporating ultrafine bubbles (UFBs) having a mean diameter of less than 1000 nm into the aqueous solution and the rate of ascorbic acid autoxidation is reduced relative to a comparable solution without UFBs, and wherein the inhibitory effect is independent of the gas species entrained within the UFBs.

In another aspect, a method to stabilize emulsions is provided. The method includes suspending ultrafine bubbles in an aqueous solution and incorporating the solution into emulsified systems, such as oil/water. The ultrafine bubbles provide added stability to the emulsion likely due to a reduction in droplet size of the oil dispersed in the water phase, and may be useful in the food, beverage and consumer products industries to extend shelf life of the products.

In yet another aspect, a method for improving spray drying efficiency and product quality by incorporating ultrafine bubbles (UFBs) into oil-in-water emulsions prior to atomization and drying is provided. The UFB-enriched emulsions demonstrate superior particle size reduction, increased surface area, enhanced stability, and produce spray-dried powders with improved encapsulation efficiency, eliminated surface oil odor, maintained particle integrity, and extended shelf life.

In a further aspect, a method for spray drying is provided. The method comprises generating ultrafine bubbles in a liquid carrier; combining the UFB-enriched carrier with material to be spray dried to form a feed material; atomizing the feed material; and drying to form powder particles; wherein the powder particles exhibit improved properties including reduced particle collapse, improved encapsulation efficiency, and/or improved particle morphology.

In another aspect, a method for spray drying oil-in-water emulsions wherein ultrafine bubble incorporation results in: at least 20% reduction in emulsion particle size; at least 40% increase in interfacial surface area; prevention of particle collapse during drying; and elimination or substantial reduction of surface oil in final powder.

In another aspect, a spray-dried powder produced by ultrafine bubble-enhanced spray drying is provided, the powder exhibiting: spherical particle morphology with smooth surfaces; reduced or eliminated surface oil content; improved encapsulation efficiency; and extended shelf life due to reduced oxidation.

In yet another aspect a method for encapsulating oils, lipids, vitamins, flavors, pharmaceuticals, or probiotics via ultrafine bubble-enhanced spray drying is provided, wherein encapsulation efficiency is improved by at least 20% and surface migration of active ingredients is reduced by at least 50% compared to conventional spray drying.

### BRIEF DESCRIPTION OF THE FIGURES

FIG. 1 illustrates a diagram of a system (101) and a method for making compositions including water and ultrafine bubbles in accordance with embodiments of the disclosure.

FIG. 2 illustrates a diagram of a system (202) and a method for making compositions including water and ultrafine bubbles utilizing a partial vacuum in accordance with embodiments of the disclosure.

FIG. 3 illustrates a diagram of multiple systems (302) and a method for making compositions including water and ultrafine bubbles in accordance with embodiments of the disclosure.

FIG. 4 illustrates a diagram of a system and method for altering the taste and mouthfeel of carbonated or gas-infused beverages by exposing a subject beverage composition containing dissolved gases to hydrodynamic cavitation in accordance with embodiments of the disclosure.

FIG. 5 illustrates a diagram of a fermentation recirculation loop system incorporating the hydrodynamic cavitation device integrated into the system.

FIG. 6 illustrates data in connection with modulation of gut microbiota compositions utilizing ultrafine bubbles. This figure illustrates relative abundance of: (a) Firmicutes and (b) Bacteroidetes populations in test (UFB water treatment) and control treatments over the course of the study. The ratio of the Firmicutes-Bacteroidetes is shown in (c). In the test treatment, the concentration of ultrafine bubble water was increased post week 6.

FIG. 7 illustrates data in connection with modulation of gut microbiota compositions utilizing ultrafine bubbles. This figure illustrates short chain fatty acid levels in the blood at (a) 8-week and (b) 12-week timepoints.

FIG. 8 illustrates data in connection with modulation of gut microbiota compositions utilizing ultrafine bubbles. This figure illustrates an overview of inflammatory markers in the test and control groups at the 12-week mark.

FIG. 9 illustrates comparison of oil slick formation in canola oil-water mixtures utilizing UFB-infused water compared to deionized water control after 90 minutes. UFB-infused water shows minimal separation and stable foam layer, while deionized water control shows complete phase separation with surface oil layer.

FIG. 10 illustrates side view comparison of canola oil mixed with ultrafine bubbles or deionized water with enhanced foam formation in ultrafine bubble treatment compared the deionized water control sample.

FIG. 11 illustrates comparison of separation indices with topical formulations made with ultrafine bubbles (1948 and 01) and without ultrafine bubbles (1948-Control & 01-Control). Lower values indicate better stability.

FIG. 12 illustrates a comparison showing impacts of using ultrafine bubble technology on emulsion stability compared to a control of deionized water without ultrafine bubbles.

FIG. 13 illustrates ultrafine bubbles (UFBs) inhibit the autoxidation of ascorbic acid in aqueous solutions.

FIG. 14 illustrates a comparison showing impacts of using ultrafine bubble technology on emulsion stability compared to a control of deionized water without ultrafine bubbles.

FIG. 15 illustrates a comparison showing particle size distributions of emulsions in oil in water slurries for spray drying using ultrafine bubbles compared to a control of deionized water without ultrafine bubbles.

FIG. 16 illustrates a comparison showing powder properties showing extent of particle collapse in emulsions used in spray drying of oil in water slurries using ultrafine bubbles compared to a control of deionized water without ultrafine bubbles.

FIG. 17 illustrates a comparison of electron scanning microscope images of emulsions used in spray drying of oil in water slurries using ultrafine bubbles compared to a control of deionized water without ultrafine bubbles.

FIG. 18 illustrates a chart showing viscosity reduction in yeast slurries using ultrafine bubbles.

### DETAILED DESCRIPTION

The present disclosure provides compositions (e.g., aqueous compositions) that comprise ultrafine bubbles, and optionally, at least one non-gaseous solute, and methods of making and using the same. The ultrafine bubbles can advantageously be used to hydrate cells and/or to dissolve, surround, or stabilize a non-gaseous solute (e.g., an organic chemical, an inorganic chemical, a protein, a peptide, a sugar, an oligosaccharide, a polysaccharide, a synthetic polymer, a fat, a wax, an oil, a colloid, a fatty acid, a DNA nucleotide, a polynucleotide, an RNA polynucleotide, a pharmaceutical drug, a fertilizer, a plant nutrient, or an electrolyte) and optionally used to deliver the solute across a cell membrane to exert its effect. As such, the disclosed compositions provide surprising and unexpected advantages in various applications, including medical and agricultural applications, based, for example, on the improved bioavailability, solubility, and/or stability. Notably, aqueous compositions that comprise a low concentration of ultrafine bubbles (e.g., at a concentration of up to 10⁸ ultrafine bubbles/mL) exert improved/increased bioavailability, solubility, permeability with respect to biological membranes, and/or stability than previously anticipated, perhaps even as compared to compositions that comprise a higher concentration of ultrafine bubbles (e.g., more than 10⁸ ultrafine bubbles/mL).

The present disclosure also relates to methods of using ultrafine bubble water compositions in various applications and methods, including but not limited to enhancement of foam stabilization and emulsion stability, modulation of gut microbiota composition, and inhibition of oxidation of ascorbic acid for use in cosmetic and other applications.

The present disclosure also provides methods of using ultrafine bubble water compositions in the modulation of gut microbiota composition, which may be useful in a variety of applications including application in non-invasive microbiome therapeutics, enhancing short chain fatty acid production for use in gut health products, reduction of inflammatory markers via microbiota modulation and therapeutic application for microbiome related conditions.

Also provided by the present disclosure are methods for making compositions comprising water and ultrafine bubbles comprising or consisting essentially of water and gases released from solution in water, optionally including steps for dissolving, surrounding, and/or stabilizing at least one non-gaseous solute with the ultrafine bubbles.

The ultrafine bubbles may be used to dissolve, surround, and/or stabilize nutrients, foods, pharmaceuticals, biologic drugs, biotechnology products, inorganic or organic chemicals. Additionally, in some embodiments, the ultrafine bubbles may be used to dissolve, surround, and/or stabilize a solute for human and/or animal nutrition, or agriculture production. In still further embodiments, the ultrafine bubbles may be used to dissolve, surround, and/or stabilize a solute in a hydration, sports, or energy drink. In other embodiments, the ultrafine bubbles may be used as a means for creating oral drug formulations with improved drug pharmacokinetics, higher drug bioavailability, increased drug safety and/or higher selective potency. Further, in other embodiments, the ultrafine bubbles may be used to dissolve, surround, and/or stabilize a drug or a biotechnology product.

In some embodiments, the disclosure provides compositions comprising ultrafine bubbles that comprise or consist essentially of water and gases released from solution in water and a non-gaseous solute, wherein the ultrafine bubbles dissolve, surround, and/or stabilize the non-gaseous solute.

In certain embodiments, the ultrafine bubbles in the composition have a median diameter of between about 2 to about 400 nanometers. In another embodiment, the ultrafine bubbles have a median diameter of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median diameter of between about 10 to about 20 nanometers or about 15 to about 20 nanometers, or about 20 to about 25 nanometers. In other embodiments, the ultrafine bubbles have a median diameter of between about 10 to about 50 nanometers, about 20 to about 50 nanometers, about 30 to about 50 nanometers, or about 40 to about 50 nanometers. In still other embodiments, the ultrafine bubbles have a median diameter of between about 50 to about 100 nanometers. In yet further embodiments, the ultrafine bubbles have a median diameter of between about 100 to about 200 nanometers, about 150 to about 200 nanometers, about 200 to about 300 nanometers, about 250 to about 300 nanometers, or about 300 to about 400 nanometers.

Without being bound by theory, it is believed the compositions as disclosed herein provide for improved bioavailability, solubility, and/or stability of the ultrafine bubbles comprising or consisting essentially of water and gases released from solution in water, as well as improved bioavailability, solubility, and/or stability of any dissolved the solutes because the ultrafine bubbles are produced from “soft” or gaseous cavitation rather than “hard” or vaporous cavitation processes. The disclosed ultrafine bubbles are believed to be (a) nucleated in the low-pressure vicinity surrounding the cavitation core, (b) sheared-off bubbles from the cavitation core itself, or (c) produced via low pressure/room temperature boiling at the core surface, such that, in the presence of turbulence and high shear stresses near the core, ultrafine bubbles are broken into smaller ultrafine bubbles through deformation (due to drag forces).

The resulting compositions incorporating ultrafine bubbles demonstrate improved efficacy in dissolving and/or stabilizing solutes, even when measured by NTA at concentrations of 10⁷ ultrafine bubbles/mL or lower. This suggests better performance at lower concentrations than expected, or possibly the presence of a sub-population of ultrafine bubbles below 50 nm that cannot be detected by NTA. Such compositions also exhibit enhanced stability over other solutions incorporating ultrafine bubbles or ultrafine bubbles produced via other means, as they can be concentrated by several orders of magnitude via rotary evaporation or crossflow filtration without ultrafine bubble loss or solute dissolution, and can even remain bottled for up to 10 years without loss of ultrafine bubble concentration or dissolution of solutes.

In cases where numerical values are indicated in the context of the present disclosure, the skilled person will understand that the technical effect of the feature in question is ensured within an interval of accuracy, which typically encompasses a deviation of the numerical value given of ±10%, and preferably of ±5%. At the very least, and not as an attempt to limit the application of the doctrine of equivalents to the scope of the claims, each numerical parameter should at least be construed in light of the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure.

Definitions of terms will be given in the following in the context of which the terms are used. The following terms or definitions are provided solely to aid in the understanding of the invention. These definitions should not be construed to have a scope less than understood by a person of ordinary skill in the art.

As used herein, an “ultrafine bubble” refers to an assembly of water molecules, with a diameter less than one micron, bonded with or otherwise associated with one another by electrostatic forces, such as hydrogen bonding, ionic bonding, van der Waals forces, or the like, surrounding gases (e.g., gases released from solution in water). In some cases, according to the disclosure, an ultrafine bubble further comprises a non-gaseous solute associated with the water molecules and dissolved within, surrounded by, and/or stabilized by the ultrafine bubble.

As used herein, a non-gaseous “solute” means a substance or particle that is fully or partially dissolved in water. In embodiments, a solute of the disclosure is dissolved within, surrounded by, and/or stabilized by ultrafine bubbles of the disclosure. A non-gaseous solute according to the disclosure comprises, without limitation, a polar or non-polar substance, a liquid, a solid, a lipid, a protein, a peptide, a nucleic acid, an organic compound, an inorganic compound, or any combination thereof.

As used herein, a “hydration agent” means, a substance that promotes the hydration of, without exclusion, a cell, a plant, a human or non-human animal, or a soil sample.

As used herein, “ultrapure water” means water prepared according to one or more of the described embodiments of the disclosure. In particular, ultrapure water refers to water prepared by methods and processes disclosed herein, or water characterized as being completely free of (e.g., does not contain any detectable amount), or substantially free of (e.g., 70%, 80%, 90%, or 95% free of), one or more impurities or contaminants.

As used herein, “bioavailability” refers to the physiological availability of a given amount of a solute as distinct from its chemical potency. For example, bioavailability refers to the proportion of an administered solute that is absorbed into the bloodstream of a mammal or is absorbed into the tissues of a plant. Bioavailability also refers to the ability of an ultrafine bubble, solute, particle, ultrafine bubble-dissolved solute, ultrafine bubble-surrounded solute, ultrafine bubble-stabilized solute, or combination thereof, to access a biological target, e.g., by crossing a biological membrane or by interacting with a biological receptor or other binding partner.

As used herein, a “microbubble” refers to an assembly of water molecules with a diameter greater than one micron and less than one millimeter, surrounding gases (e.g., generated gases added or bubbled into a water source).

### Compositions Comprising Ultrafine Bubbles

The disclosure provides compositions and solutions comprising ultrafine bubbles (e.g., ultrafine bubbles comprising or consisting essentially of water and gases released from solution in water), and optionally a non-gaseous solute dissolved, surrounded, and/or stabilized by the ultrafine bubbles, wherein the ultrafine bubbles are present in the composition at a concentration of up to 10¹² ultrafine bubbles/mL (e.g., at a range of 10 to 10² ultrafine bubbles/mL, 10² to 10³ ultrafine bubbles/mL, 10³ to 10⁴ ultrafine bubbles/mL, 10⁴ to 10⁵ ultrafine bubbles/mL, 10⁵ to 10⁶ ultrafine bubbles/mL, 10⁶ to 10⁷ ultrafine bubbles/mL, or 10⁷ to 10⁸ ultrafine bubbles/mL, or 10⁸ to 10⁹ ultrafine bubbles/mL, or 10⁹ to 10¹⁰ ultrafine bubbles/mL, or 10¹⁰ to 10¹¹ ultrafine bubbles/mL, or 10¹¹ to 10¹² ultrafine bubbles/mL).

In some embodiments, the ultrafine bubbles may have a median size of between about 2 to about 400 nanometers.

The water may be prepared by processes known in the art and used as a starting material for generating the compositions and solutions comprising ultrafine bubbles as disclosed herein. The water may be prepared by carbon filtration, by slow sand filtration, by reverse osmosis, by electro-deionization treatment, by ultraviolet light exposure, or by a combination comprising two or more of the processes described herein. For example, the water may be prepared by a sequential process comprising each of carbon filtration, slow sand filtration, reverse osmosis, electro-deionization treatment, and ultraviolet light exposure. Alternatively, the water may be prepared according to one or more of the processes described herein in combination with other methods of water purification known in the art but not expressly recited herein.

The water may be prepared by a process comprising the steps of: filtering a volume of water with a carbon filter to produce an amount of water with a low chlorine content; removing ions in the carbon filtered water by a reverse osmosis process to produce a supply of a deionized water; electro-deionizing the supply of the deionized water from the reverse osmosis process to make a water supply; testing the resistivity of the water to determine if the resistivity of the water is between about 17 meg-ohm cm to about 18.2 meg-ohm cm; repeating a process step for preparing the water and retesting the resistivity of the water until the water has a measured resistivity of between about 17 meg-ohm cm to about 18.2 meg-ohm cm; irradiating the supply of the water having a measured resistivity of between about 17 meg-ohm cm to about 18.2 meg-ohm cm with ultraviolet light to make a sterilized water supply; and storing the sterilized water in a stainless steel container until sterilized water is needed to be added in the process to make an composition comprising ultrafine bubbles.

The water may be purified of contaminants including, for example, organic and inorganic compounds; dissolved and particulate matter; volatile and non-volatile matter, reactive and inert matter; hydrophilic and hydrophobic matter.

The water of the disclosure may have a high oxidative reduction potential including, for example, about 80 to about 600 mV. Further, the pH of the water may be between about 3 to about 7, preferably about 4 to about 6 and the resistivity of the water may be between about 17 to about 18.2 meg-ohm cm.

In some embodiments, the compositions comprise ultrafine bubbles comprising or consisting essentially of water and gases released from solution in water, wherein the ultrafine bubbles optionally dissolve, surround, and/or stabilize a non-gaseous solute, and wherein the water has an oxidative reduction potential between about −200 mV and 800 mV.

In some embodiments, the compositions comprise ultrafine bubbles comprising or consisting essentially of water and gases released from solution in water, wherein the ultrafine bubbles optionally dissolve, surround, and/or stabilize a non-gaseous solute, and wherein the composition has a zeta potential of between about absolute value 0 and 40. In further embodiments, the zeta potential of the composition is between about −40 mV to about 0 mV, although in most embodiments the average zeta potential falls between −15 to −10 mV. In still further embodiments, the zeta potential of the composition is between about −40 mV to about −35 mV, about −35 to about −30 mV, about −30 to about −25 mV, about −25 to about −20 mV, about −20 mV to about −15 mV, about −15 mV to about −10 mV, about −10 mV to about −5 mV, about −5 mV to about 0 mV. The inventors have surprisingly found that despite relatively low average absolute value zeta potentials of the compositions (e.g., typical ultrafine bubble compositions have zeta potentials of around absolute value 30 mV, significantly higher than the average absolute value zeta potentials of the compositions herein), the ultrafine bubble compositions according to the disclosure herein achieve superior stability results over ultrafine bubbles formed by alternative means with higher absolute value zeta potentials.

A non-gaseous solute dissolved within, surrounded by, and/or stabilized by ultrafine bubbles (e.g., ultrafine bubbles that comprise or consist essentially of water and gases released from solution in water) may be a small molecule drug, a protein, a peptide, or a combination thereof. In some embodiments, the non-gaseous solute comprises a cellular detoxification agent, a hydration agent, an anti-inflammatory agent, a neuroprotective agent, a neuromodulatory agent, or an anti-tumorigenic agent. In still other embodiments, the non-gaseous solute improves ATP production.

The ultrafine bubbles (e.g., ultrafine bubbles that comprise or consist essentially of water and gases released from solution in water) have a median diameter of between about 2 to about 400 nanometers. In certain embodiments, the ultrafine bubbles have a median diameter of about 1 nanometers, about 2 nanometers, about 3 nanometers, about 4 nanometers, about 5 nanometers, about 6 nanometers, about 7 nanometers, about 8 nanometers, about 9 nanometers, about 10 nanometers, about 11 nanometers, about 12 nanometers, about 13 nanometers, about 14 nanometers, about 15 nanometers, about 16 nanometers, about 17 nanometers, about 18 nanometers, about 19 nanometers, or about 20 nanometers. In other embodiments, the ultrafine bubbles according to the disclosure comprise a median diameter of about 20 nanometers, about 22 nanometers, about 24 nanometers, about 26 nanometers, about 28 nanometers, or about 30 nanometers. In still other embodiments, the ultrafine bubbles according to the disclosure comprise a median diameter of about 35 nanometers, about 40 nanometers, about 45 nanometers, about 50 nanometers, about 60 nanometers, about 70 nanometers, about 80 nanometers, about 90 nanometers, or about 100 nanometers. In other embodiments, the ultrafine bubbles according to the disclosure comprise a median diameter of about 110 nm, about 120 nm, about 130 nm, about 140 nm, about 150 nm, about 160 nm, about 170 nm, about 180 nm, about 200 nm, about 210 nm, about 220 nm, about 230 nm, about 240 nm, about 250 nm, about 260 nm, about 270 nm, about 280 nm, about 300 nm, 310 nm, about 320 nm, about 330 nm, about 340 nm, about 350 nm, about 360 nm, about 370 nm, about 380 nm, or about 400 nm.

In some embodiments, the ultrafine bubbles comprise on average about 20, about 25, about 30, about 35, about 40, about 45, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500 water molecules. In other embodiments, the ultrafine bubble comprises between about 50 and about 100 water molecules, about 100 to about 150 water molecules, about 150 to about 200 water molecules, about 200 to about 250 water molecules, about 250 to about 300 water molecules, about 300 to about 350 water molecules, about 350 to about 400 water molecules, about 400 to about 450 water molecules, or about 450 to about 500 water molecules.

In some embodiments, the ultrafine bubbles fully dissolve, surround, and/or stabilize a non-gaseous solute. In other embodiments, the ultrafine bubbles substantially dissolve, surround, and/or stabilize a non-gaseous solute (e.g., dissolve, surround, and/or stabilize about 50%, 55%, 60%, 65%, 70%, 80%, 85%, 90%, 95% or more of the non-gaseous solute).

Those skilled in the art will recognize different ways of measuring particle size distribution of an ultrafine bubble suspension of the disclosure. In an exemplary method a particle size distribution of an ultrafine bubble suspension is measured using a Malvern Instruments Zetasizer Nano ZSP, which is a high-performance system and particularly suitable for the characterization of ultrafine bubbles. Alternatively, the concentration and particle size distribution of an ultrafine bubble suspension of the disclosure may be measured using a Malvern Instruments Nanosight NTA instrument. In another exemplary method a diameter of an ultrafine bubble is measured using liquid-cell transmission electron microscopy (TEM). Additionally, the size distribution and concentration of an ultrafine bubble suspension may be measured on a particle-by-particle basis using tunable resistive pulse sensing (TRPS) or electrical zone sensing, using such instruments as the Izon Exoid or the Beckman Coulter Multisizer 4e, respectively.

In some embodiments, the ultrafine bubble and solutes of the disclosure are measured according to the following non-limiting parameters: ultrafine bubble diameter, particle and molecule size, translational diffusion, electrophoretic mobility, zeta potential of particles at high and low concentrations, viscosity and viscoelasticity of protein and polymer solutions, concentration, and/or molecular weight (e.g., kD).

In some embodiments, the ultrafine bubbles (e.g., ultrafine bubbles comprising or consisting essentially of water and gases released from solution in water) are stable for an extended storage period including, for example, a period of years. In some embodiments, the ultrafine bubbles are stable for about 2 years, about 2.5 years, about 3 years, about 4 years, about 5 years, about 6 years, about 7 years, about 8 years, about 9 years, or about 10 years. In some embodiments, the ultrafine bubbles are stable for a period in excess of 10 years.

In some embodiments, the ultrafine bubbles dissolve, surround, and/or stabilize a non-gaseous solute for a period of years, for example for about 2 years, about 2.5 years, about 3 years, about 4 years, about 5 years, about 6 years, about 7 years, about 8 years, about 9 years, or about 10 years. In further embodiments, the ultrafine bubbles dissolve, surround, and/or stabilize a non-gaseous solute for a period in excess of 10 years.

In some embodiments, the disclosure provides compositions or solutions for use in delivering a non-gaseous solute to the interior of a cell such as a plant or animal (e.g, mammalian including human cell). In other embodiments, the disclosure provides compositions or solutions for use in delivering a non-gaseous solute to the interior of a plant or an animal cell.

Embodiments of the disclosure include compositions or solutions wherein a non-gaseous solute is dissolved within, surrounded by, and/or stabilized by ultrafine bubbles and has improved bioavailability relative to a composition or a solution where the non-gaseous solute is not dissolved by, surrounded by, and/or stabilized by ultrafine bubbles. In some embodiments, the non-gaseous solute dissolved within, surrounded by, and/or stabilized by ultrafine bubbles has improved bioavailability by virtue of its ability to access the interior of a cell. For example, a water having a non-gaseous solute is typically incapable of passing through a cell membrane, but non-gaseous solutes dissolved within, surrounded by, and/or stabilized by the ultrafine bubbles of the disclosure are able to cross a cell membrane; i.e., they increase cell permeability. In some embodiments, a cell membrane may be a plasma membrane, a nuclear membrane, a cell wall, or any other impermeable barrier defining the boundaries of a cell or an organelle within a cell.

In other embodiments, a non-gaseous solute dissolved within, surrounded by, and/or stabilized by ultrafine bubbles has improved bioavailability by virtue of its ability to access an intracellular space. In still other embodiments, the solute dissolved within, surrounded by, and/or stabilized by ultrafine bubbles has improved bioavailability by virtue of its ability to access specific plant or animal tissue types, such as root or leaf tissue in a plant, or skin or internal organ tissues in an animal. In yet other embodiments, an ultrafine bubble comprising or consisting essentially of water and gases released from solution in water and gases released from solution in the ultrapure water has improved bioavailability relative to an ultrafine bubble that does not comprise ultrapure water.

In some embodiments, the compositions including ultrafine bubbles and dissolved, surrounded, and/or stabilized non-gaseous solutes have improved bioavailability relative to naturally occurring water and dissolved solutes. In some embodiments, the ultrafine bubbles and dissolved, surrounded, and/or stabilized non-gaseous solutes provided herein render an otherwise unavailable non-gaseous solute bioavailable, in which case the disclosure provides improved bioavailability of the solute relative to the non-gaseous solute that is not dissolved by, surrounded by, and/or stabilized by ultrafine bubbles. In other embodiments, the ultrafine bubbles comprising or consisting essentially of water and gases released from solution in water, wherein the ultrafine bubbles dissolve, surround, and/or stabilize a non-gaseous solute improve bioavailability of the solute by about 10%, about 20%, about 30%, about 40%, about 50%, about 60%, about 70%, about 80%, or about 90% relative to the solute that is not dissolved within, surrounded by, and/or stabilized by ultrafine bubbles. In further embodiments, the ultrafine bubbles comprising or consisting essentially of water and gases released from solution in water and dissolved, surrounded, and/or stabilized non-gaseous solutes improve bioavailability of the solute by about 1%, about 2%, about 3%, about 4%, about 5%, about 6%, about 7%, about 8%, or about 9%.

In some embodiments, the disclosure provides methods for improving the bioavailability of a non-gaseous solute, including, for example, dissolving the solute in water and dissolving, surrounding, and/or stabilizing the solute with the ultrafine bubbles disclosed herein, wherein the ultrafine bubble has a median diameter between about 2 to about 400 nanometers.

In some embodiments, the compositions having a solute dissolved within, surrounded by, and/or stabilized by ultrafine bubbles have improved stability relative to compositions having the solute that is not dissolved within, surrounded by, and/or stabilized by ultrafine bubbles. In some embodiments, the solute dissolved within, surrounded by, and/or stabilized by ultrafine bubbles with improved stability has an increased half-life, such as an increased serum half-life or solution half-life. In some embodiments, the solute dissolved within, surrounded by, and/or stabilized by ultrafine bubbles has improved stability for extended storage periods relative to the solute that is not dissolved within, surrounded by, and/or stabilized by ultrafine bubbles.

In some embodiments, the compositions or solutions including ultrafine bubbles and a solute dissolved within, surrounded by, and/or stabilized by ultrafine bubbles have improved solubility relative to compositions or solutions including the solute that is not dissolved within, surrounded by, and/or stabilized by ultrafine bubbles. In other embodiments, a solute dissolved within, surrounded by, and/or stabilized by ultrafine bubbles comprises a drug with increased solubility, improved pharmacokinetics, and/or increased bioavailability. As such, embodiments of the disclosure have applications where improved solubility, pharmacokinetics, and/or bioavailability is desired, for example, without limitation, in medical products, patient care, medical research, medical testing, medical equipment, cell culture, and surgical procedures.

In some embodiments, the solute dissolved within, surrounded by, and/or stabilized by ultrafine bubbles normally has limited or no solubility in water but is solubilized when dissolved within, surrounded by, and/or stabilized by ultrafine bubbles. In alternative embodiments, the solute dissolved within, surrounded by, and/or stabilized by ultrafine bubbles may have low to moderate solubility in water but is solubilized (e.g., completely solubilized) when dissolved within, surrounded by, and/or stabilized by ultrafine bubbles comprising or consisting essentially of water and gases released from solution in water.

In some embodiments, a non-gaseous solute of the disclosure further comprises a surface coating applied before or after dissolving, surrounding, and/or stabilizing the solute with ultrafine bubbles. For biological applications, such as proteins, the surface coating may be polar to give high aqueous solubility and prevent particle aggregation.

### Process for Making Compositions Comprising Ultrafine Bubbles

The present disclosure also provides a process of making a composition or solution comprising water and ultrafine bubbles that may optionally dissolve, surround, and/or stabilize a solute. In some embodiments, a water supply is subjected to a combination of hydrodynamic cavitation, shear forces, and low pressure/room temperature boiling to form ultrafine bubbles, and the formed ultrafine bubbles from the water supply are added to the water of the composition. In some embodiments, a water supply is subjected to processing that forms ultrafine bubbles via gaseous cavitation, and the formed ultrafine bubbles from the water supply are added to the water of the composition.

In another aspect disclosed herein, a method for producing a composition comprising water and ultrafine bubbles (e.g., ultrafine bubbles including gases released from solution in water) is provided. The method includes subjecting water to a combination of hydrodynamic cavitation, shear forces, and low pressure/room temperature boiling to produce ultrafine bubbles formed by release of dissolved gases from the water. In some embodiments, the water is selected from DI water, ultrapure water, tap water, groundwater (e.g., well water), surface water, and reverse osmosis water. In particular embodiments, the water is ultrapure water. In some embodiments, the water is tap water.

The method may comprise one or more (including all) of the following steps: adding water to a tank; pumping the water at a selected flow rate through a transfer pipe from the tank to a nozzle with one jet opening or a plurality of jet openings inside a hollow cylinder; using the one jet opening or the plurality of jet openings in the nozzle to jet the water into the hollow cylinder; wherein the selected flow rate creates a vortex of the water inside the hollow cylinder, thereby subjecting the water to a combination of hydrodynamic cavitation, shear forces, and thin film boiling to produce ultrafine bubbles formed by release of dissolved gases from the water (i.e., gaseous cavitation). The process according to certain embodiments may further comprise collecting the composition comprising the water and ultrafine bubbles; and using the ultrafine bubbles of the composition to dissolve, surround, and/or stabilized a non-gaseous solute to improve the bioavailability of the solute.

In some embodiments, a water supply is subjected to a combination of hydrodynamic cavitation, shear forces, and low pressure/room temperature boiling to form ultrafine bubbles, and the formed ultrafine bubbles from the water supply are added to water to form the composition. In some embodiments, a water supply is subjected to processing that forms ultrafine bubbles via gaseous cavitation, and the formed ultrafine bubbles from the water supply are added to water to form a composition as set forth herein. In other embodiments, ultrafine bubbles comprising water and gases released from solution in a first water source are added to a second water source to make compositions as set forth herein. In some embodiments, a non-gaseous solute is added to the composition including the formed ultrafine bubbles and the water supply to dissolve, surround, and/or stabilize the non-gaseous solute with the formed ultrafine bubbles.

The present disclosure also provides methods for dissolving, surrounding, and/or stabilizing a non-gaseous solute with ultrafine bubbles comprising or consisting essentially of water and gases released from solution in water.

In some embodiments, the disclosure provides a process for dissolving, surrounding, and/or stabilizing a non-gaseous solute with ultrafine bubbles comprising or consisting essentially of water and gases released from solution in water, the process comprising: selecting an amount of solute to add to a volume of water; combining the solute and water in a mixing tank to form a blended aqueous composition; pumping the blended aqueous composition at a selected flow rate through a transfer pipe from the mixing tank to a nozzle with one jet opening or a plurality of jet openings inside a hollow cylinder; using the one jet opening or the plurality of jet openings in the nozzle to jet the blended aqueous composition into the hollow cylinder; wherein the selected flow rate creates a vortex of the blended aqueous composition inside the hollow cylinder that dissolve, surround, and/or stabilizes the solutes and reduce sizes of the ultrafine bubbles in the blended aqueous composition. The process according to certain embodiments may further comprise collecting the composition comprising the solute dissolved within, surrounded by, and/or stabilized by the ultrafine bubbles; and using the reduced size ultrafine bubbles dissolving, surrounding, and/or stabilizing the solute to improve the bioavailability of the solute.

In some embodiments, a process is provided for reducing the size of ultrafine bubbles in a solution of water substantially free of dissolved non-gaseous solutes comprising pumping water at a selected flow rate through a transfer pipe to a nozzle with one jet opening or a plurality of jet openings inside a hollow cylinder; using the one jet opening or the plurality of jet openings in the nozzle to jet the blended composition into the hollow cylinder; wherein the selected flow rate creates a vortex of the blended composition inside the hollow cylinder that reduces the size of the ultrafine bubbles in the blended composition.

In another aspect disclosed herein, a method for producing a composition comprising water and ultrafine bubbles including gases released from solution in water is provided. The method includes subjecting water to a combination of hydrodynamic cavitation, shear forces, and low pressure/room temperature boiling to produce ultrafine bubbles formed by release of dissolved gases from the water. In some embodiments, the water is selected from DI water, ultrapure water, tap water, groundwater (e.g., well water), surface water, and reverse osmosis water. In particular embodiments, the water is ultrapure water.

In some embodiments of the methods, the methods further comprise concentrating the ultrafine bubbles within the composition via rotary evaporation or cross flow filtration.

In another aspect disclosed herein, the disclosure relates to a novel formulation method designed to preserve ultrafine bubbles indefinitely within a solution. The formulation consists of an ultrafine bubble suspension combined with an ingredient that increases the viscosity of the final product. By enhancing the viscosity, the mobility of the ultrafine bubbles is restricted, effectively preventing them from bursting or coalescing. This stabilization mechanism ensures that the ultrafine bubbles remain uniformly dispersed within the solution over extended periods, making the formulation suitable for various applications, including skincare, pharmaceutical, and consumable products.

The key innovation lies in the use of viscosity modifiers that interact with the ultrafine bubbles, creating a stable environment that counters the natural tendencies of these bubbles to merge or collapse. The result is a formulation with enhanced shelf life and consistent performance, providing long-lasting efficacy in delivering active ingredients or maintaining the structural integrity of the product.

Viscosity increasing compounds commonly used in food and cosmetic formulations to increase viscosity include: polysaccharides, including xanthan gum, guar gum, carrageenan, agar-agar, alginate, pectin; cellulose derivatives including hydroxyethylcellulose (HEC), carboxymethylcellulose (CMC), methylcellulose; proteins, including gelatin, collagen, whey protein concentrate; synthetic polymers including carbomers, polyacrylamide, polyvinyl alcohol (PVA), and polyethylene glycol (PEG); natural gums and resins, including, gellan gum, Arabic gun. Tragacanth gum, and vegetable glycerin; lipids and waxes including beeswax, steric acid, cetyl alcohol; starches including corn starch, potato starch, tapioca starch and silicates, including magnesium aluminum silicate, bentonite clay. The formulation includes an ultrafine bubble suspension and a viscosity-increasing ingredient, wherein the ultrafine bubbles are preserved indefinitely within the solution due to inhibited bursting or coalescence. Additionally, the method of stabilizing ultrafine bubbles in a solution includes incorporating a viscosity-increasing agent, thereby preventing the coalescence or bursting of the bubbles, ensuring long-term stability and efficacy of the formulation. The present disclosure further includes skincare or consumable products formulated with an ultrafine bubble suspension and a viscosity modifier, wherein the ultrafine bubbles remain stable and uniformly dispersed for extended periods, enhancing the product's performance and shelf life, and methods for preparing a stable ultrafine bubble suspension in a viscous medium, wherein the increased viscosity prevents bubble coalescence, enabling the formulation's use in diverse applications, including topical, oral, or other administration forms.

This disclosure is further illustrated by the following examples which are provided to facilitate the practice of the disclosed methods. These examples do not limit the scope of the disclosure in any way.

### EXAMPLES

### Example 1: Methods of Making Compositions Including Water and Ultrafine Bubbles

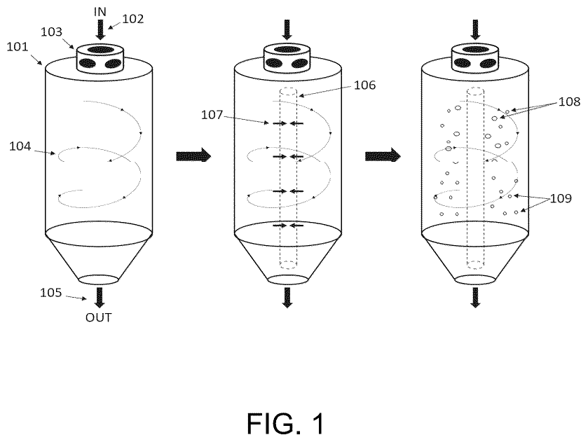

With reference to FIG. 1, a system (101) and a process for making compositions including water and ultrafine bubbles in accordance with embodiments of the disclosure is provided (herein after known as “Hydrosome Technology”). Water enters the vortexing/hydrodynamic cavitation system (101) at step (102) via the nozzle (103) and imparts a vortex flow (104). The vortex core (106) forms as dissolved gases are drawn out of solution due to low pressure at the center (107). Without being bound by theory, it is believed micro- and ultrafine bubbles form (108) spontaneously due to low pressures near to core surface, due to gas being sheared from the core surface, and/or due to room-temperature low pressure boiling at the core surface. Shear and drag forces are believed to break the microbubbles into ultrafine bubbles resulting in a near uniform size distribution (109). The resulting composition including water and ultrafine bubbles flows from the system (101) via the exit (105).

In an embodiment of the invention, a non-gaseous solute (e.g., zinc sulfate) is added to the water prior to its entry to the system 101 at step 102, and the resulting composition including water, ultrafine bubbles, and the non-gaseous solute (e.g., zinc sulfate) flows from the system (101) via the exit (105). The ultrafine bubbles of the composition dissolve, surround, and/or stabilize the non-gaseous solute.

In another embodiment of the invention, a non-gaseous solute (e.g., zinc sulfate) is added to a composition including water and ultrafine bubbles after the composition exits from the system (101) via the exit (105). The ultrafine bubbles of the composition dissolve, surround, and/or stabilize the non-gaseous solute.

In another embodiment of the invention, the vortexing/hydrodynamic cavitation system (101) is used to produce an ultrafine bubble suspension or composition comprising water and ultrafine bubbles. The ultrafine bubbles from the ultrafine bubble suspension or composition comprising water and ultrafine bubbles are then added to a different source of water.

In another embodiment of the invention, the water is “enriched” with microbubbles (bubbles greater than one micron and less than a millimeter in diameter) prior to entering the system (101) via the nozzle (103). These bubbles may be added from an exogenous source such as a microbubble generator, venturi, or porous bubbler/membrane in-line or into a tank before processing. The resulting composition exiting via the exit (105) may have higher concentrations of ultrafine bubbles as a result (e.g., greater than 10⁸ ultrafine bubbles/mL). In some embodiments, the compositions have between about 10⁸ ultrafine bubbles/mL and 10¹² ultrafine bubbles/mL. Without being bound by theory, it is believed this is due to the breakup of the microbubbles into ultrafine bubbles while passing through the system (101) as the microbubbles are exposed to drag forces. Furthermore, by creating microbubbles from specific gases, particularly gases that do not readily dissolve into water (e.g. ozone), the composition of the resulting ultrafine bubbles may be controlled or tailored to include a wider range of gases than are present in the atmosphere.

In another embodiment of the invention, the water is sparged with one or more specific gases prior to entering the system (101) via the nozzle (103). In some embodiments, the resulting composition of gases contained within the ultrafine bubbles is tailored. In some embodiments, the sparging gases may include, but not be limited to, one or more of O₂, O₃, CO₂, N₂, N₂O, Ar, or any mixture containing those gases. For example, when O₂ gas and N₂ gas are sparged or bubbled in water in order to saturate the water prior to undergoing the process within system (101), the resulting composition will have a higher concentration of O₂ and N₂ ultrafine bubbles than if the water had only been exposed to the atmosphere. Such a resulting composition may have particular benefits, such as increasing plant growth upon application.

In another embodiment of the invention, enrichment with large ultrafine bubbles (>100 nm) is carried out using various methods before entering the vortexing/hydrodynamic cavitation system (101). These methods, such as pressurization, gas injection, or mechanical agitation, introduce these larger bubbles into the solution. During the subsequent treatment, these large bubbles are then broken down into smaller ones (<100 nm) through hydrodynamic cavitation and shear. This dual-stage process not only increases the overall concentration of bubbles in the solution but also allows for precise customization of bubble compositions.

The implications of this advancement are significant. By starting with larger bubbles and refining them into smaller ones through cavitation and shear forces, this method provides a more controlled and efficient means of bubble manipulation. This level of control enables the tailoring of bubble properties to match specific application requirements, such as in biomedical applications, environmental remediation, or industrial processes. Additionally, the increased concentration of smaller bubbles enhances the effectiveness of bubble-based treatments, potentially leading to improved outcomes and efficiency in various fields of application.

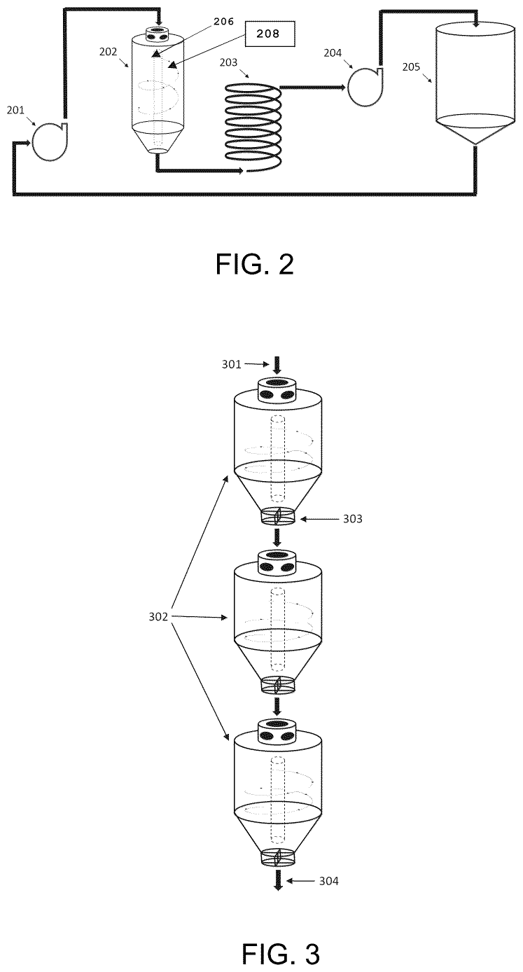

In another embodiment of the disclosure shown in FIG. 2, after creating ultrafine bubbles through vortexing/hydrodynamic cavitation, these bubbles, along with possibly the vortex core (206), are kept under a partial vacuum for some time. This is done by placing the suction side of a pump (204) at the outlet of the cavitation device (202). The partial vacuum helps maintain the stability and concentration of the bubble suspension (208). Adjusting the concentration of the bubble suspension (208) is accomplished by controlling either how long the bubble suspension stays in the vacuum (203) or the distance it travels through the vacuum. Moreover, the vortex core (206) may continue into the dwell tubing beyond the technical exit. This method permits customization of the properties of the suspension for specific needs, by utilizing either a single self-priming pump (204) (like a positive displacement pump) after vortexing/hydrodynamic cavitation treatment or using two pumps in a push-pull setup with a first pump (201) before vortexing/hydrodynamic cavitation treatment and a second pump (204) after the vortexing/hydrodynamic cavitation treatment, respectively.

In another embodiment of the invention, highly concentrated bubble suspensions are achieved by circulating the ultrafine bubble suspension through the hydrodynamic cavitation device (202) and suction pump (201), (204) multiple times using a reservoir (205) as described above for FIG. 2.

FIG. 3 illustrates an alternative approach for creating highly concentrated bubble suspensions, which involves passing water (301) through multiple vortexing/hydrodynamic cavitation treatment devices (302) set up in a series or sequence. To achieve this, arrestors (303) are utilized to halt the flow rotation of the bubble suspension at the end of each treatment, while multiple pumps (201), (204) are utilized to sustain the vacuum. This sequential setup or recirculation approach provides scalability and efficiency in generating highly concentrated bubble suspensions, reaching up to 10¹¹ bubbles per milliliter.

### Example 2: Hydrodynamic Cavitation for Enhancing Mouthfeel in Carbonated and Gas-Infused Beverages

Background: Carbon dioxide was injected between the prime pump and the ultrafine bubble-generating device as described above resulting in a distinct effect on taste compared to water infused with the same concentration of carbon dioxide under otherwise identical conditions. Despite having equal levels of dissolved carbon dioxide, the two water samples produced noticeably different sensory experiences. Water carbonated and processed through the present device and system exhibited a unique mouthfeel, with reduced effervescence targeting the back of the tongue rather than the tip and imparting a citrus-like taste.

While ultrafine bubbles may contribute to this phenomenon, it is likely the hydrodynamic cavitation occurring during the ultrafine bubble generation process is the primary factor. Without being bound by theory, this effect may result from alterations in the ratio of dissolved carbon dioxide to carbonic acid in the water, potentially induced by the hydrodynamic cavitation process. This approach offers a valuable application for flavorists, enabling the modification of taste, flavor, and mouthfeel in carbonated or gas-infused beverages without relying on additives.

### Summary of Experiment

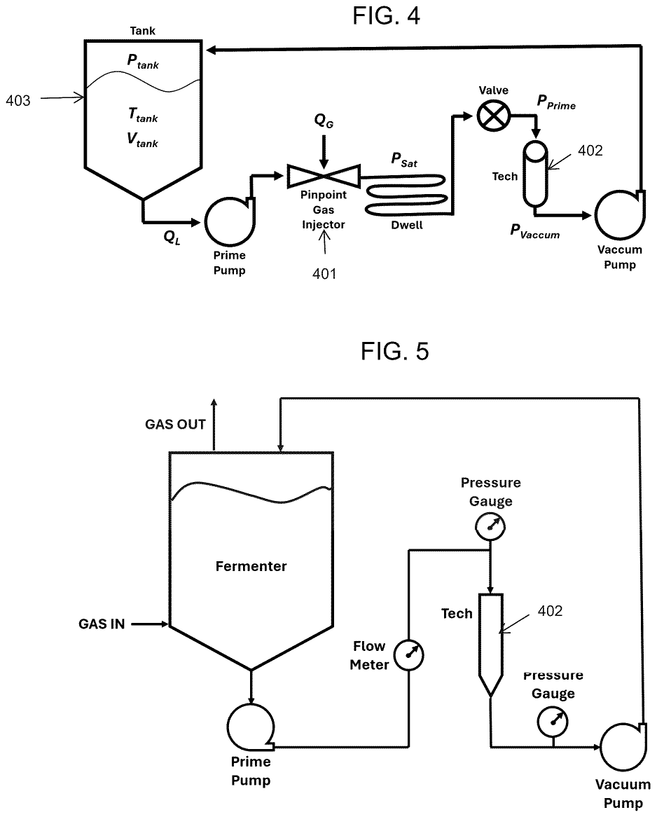

Test samples were created by introducing carbon dioxide into chilled deionized water using a pinpoint carbonator (401) and allowing it to dissolve under high pressure (FIG. 4). The carbonation process was initiated by closing the valve shown in FIG. 4, allowing pressure to build until the saturation pressure reached at least 30 psi. This ensured that all injected gas was effectively dissolved while passing through an extended dwell tube, where additional residence time facilitated complete dissolution.

Once the desired pressure was reached, the carbonated water was directed through a vortexing/hydrodynamic cavitation device (402). This step served to disrupt and further refine the dissolved gas-liquid interface, promoting uniform distribution of dissolved CO₂ while potentially generating a population of ultrafine bubbles. The processed water was then pumped into a pressurized tank (403) kept at approximately 15 psi for storage and further analysis. The infusion of carbon dioxide continued until the concentration of dissolved gases reached 2.0 volumes.

For the control, the above procedure was repeated without the use of the vortexing/hydrodynamic cavitation device in the system, producing carbonated water with an identical dissolved carbon dioxide concentration of 2.0 volumes. Both water samples, the test sample and the control sample, were then subjected to single-blind taste testing. Panelists reported notable differences between the test and control samples. The effervescence of the test samples was described as less prominent, less sharp, and more targeted toward the back of the tongue. Additionally, some panelists noted a citrus-like quality in the test samples. Therefore, the process supports the proposal of enabling modification of taste, flavor, and mouthfeel in carbonated or gas-infused beverages using ultrafine bubbles.

### Examples 4-8—Effects of Ultrafine Bubble Water on Gut Microbiota in Rats

Experimental design for the following examples is detailed in the peer-reviewed publication: “Effects of Ultrafine Bubble Water on Gut Microbiota Composition and Health Markers in Rats” (Nanomaterials 2025, 15, 1193), authored by the inventors in the present application and incorporated by reference herein in its entirety. The study design utilized twenty-four female Sprague Dawley rats over a twelve-week period. Twelve rats in the control group received deionized water, while twelve rats in the test group received ultrafine bubble water. Air-filled UFB water was generated via a patent-pending hydrodynamic cavitation process (U.S. Patent Application Publication No. 2025/0161890 which is incorporated herein by reference in its entirety).

Following a 14-day acclimatization period, the rats were randomized into two groups of twelve animals each: the control group received deionized water, while the test group received UFB water. Water bottles were refilled every two days or as needed. UFB concentrations in the test group were intentionally increased at week 7 to investigate dose-dependent effects on biological parameters. Blood samples and fresh fecal samples for microbiome analysis were collected at weeks 0, 6, 8, and 12.

### Example 4: Modulation of Gut Microbiota Composition Via Ultrafine Bubble Water

Administration of air-filled ultrafine bubble water in drinking water induces systematic shifts in gut microbiome populations. Specifically increases Bacteroidetes (+122%) and decreases Firmicutes (−43%) compared to controls, with effects demonstrating concentration-dependent threshold responses were noted. Bacteroidetes and Firmicutes represent the dominant gut bacterial populations and their ratio indicates metabolic health status.

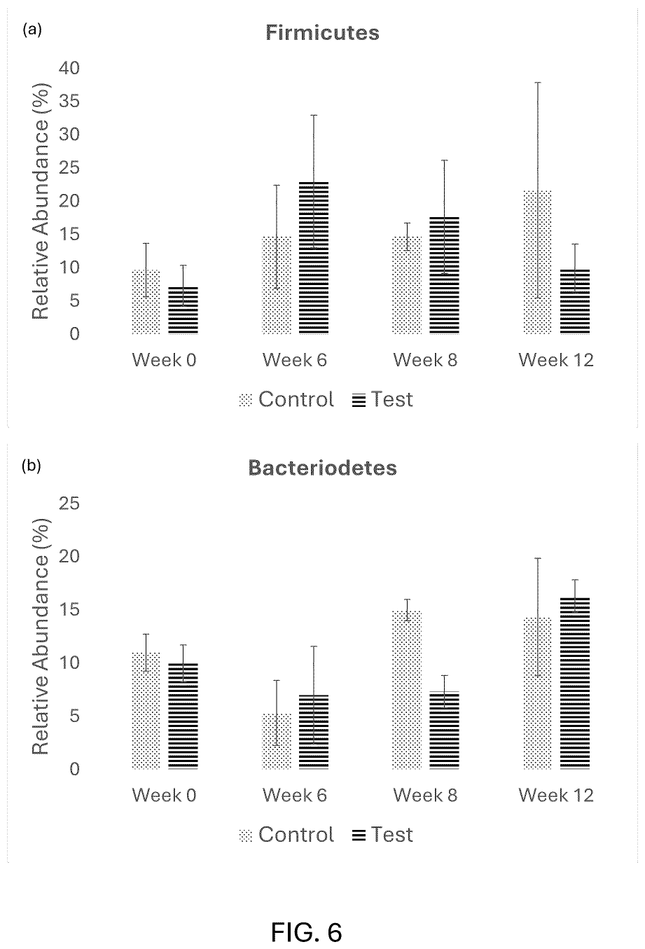

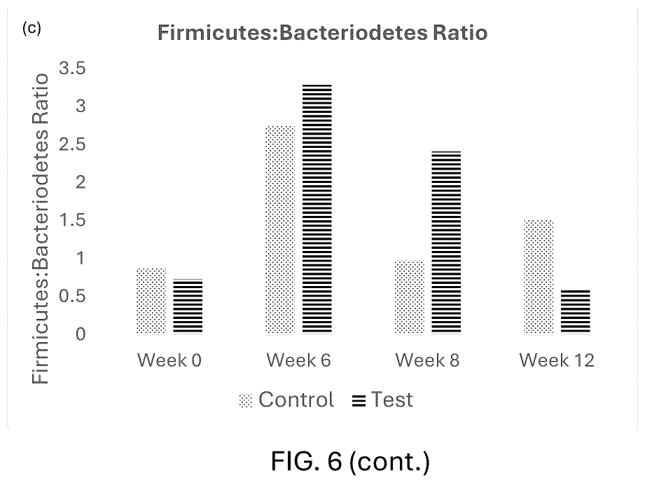

FIG. 6 illustrates the key results of the above study, specifically increases in Bacteroidetes (+122%) and decreases in Firmicutes (−43%) compared to controls, with effects demonstrating concentration-dependent threshold responses.

### Example 5: Enhancement of Short-Chain Fatty Acid (SCFA) Production

This example demonstrates ultrafine bubble water treatment significantly increases production of beneficial short chain fatty acids (SCFAs) through microbiome modulation. Effects are sustained over 12-week period and selective to specific SCFA pathways, indicating stable metabolic modification.

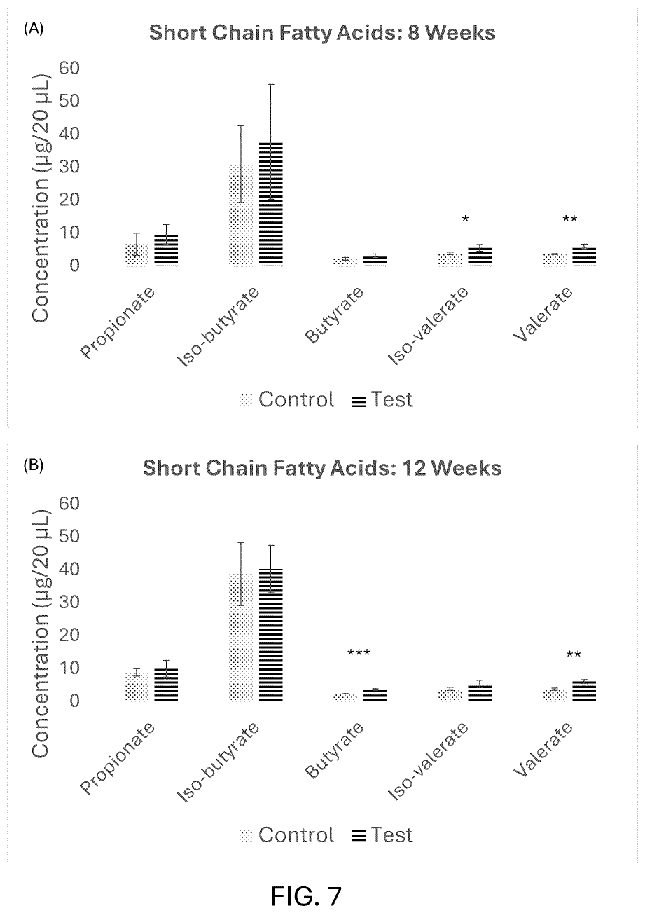

Treatment with UFB water (test group) induced substantial changes in the SCFA production profiles (FIG. 7). Analysis in week 8 revealed significant increases across multiple SCFA metabolites in the test group compared to the control group. Iso-valerate levels increased by 45.3% (p≤0.05), and valerate increased by 66.3% (p≤0.01). These elevations were largely maintained throughout week 12, with butyrate (p≤0.001) and valerate (p≤0.01) showing the most sustained increases at 56.0% and 63.1%, respectively. Acetate levels remained below detection limits in both groups throughout the study period, suggesting specific modulations of SCFA metabolic pathways rather than a general increase in fermentation activity.

### Example 6: Reduction of Inflammatory Markers Via Microbiome Modulation

This example demonstrates comprehensive downregulation of inflammatory markers through microbiome-mediated pathways. Concurrent reduction in pro-inflammatory (TNF-α, IL-1β) and regulatory (IL-10) cytokines indicates complex immune environment remodeling associated with microbiome composition changes.

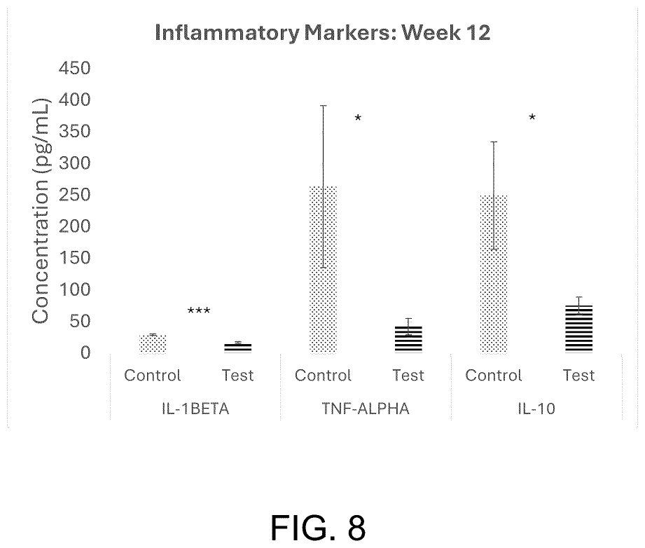

The inflammatory profile was significantly altered in response to the test group (FIG. 8). By week 12, substantial reductions were observed in both pro-inflammatory and regulatory cytokines. IL-1β levels decreased by 41.0% (p≤0.05) in the test group compared to the control group, while TNF-α showed the most dramatic reduction of 84.0% (p≤0.05). Notably, the regulatory cytokine IL-10 also decreased by 69.8% (p≤0.05). This comprehensive downregulation of inflammatory markers suggests a broad modulation of immune response pathways rather than a simple anti-inflammatory effect.

### Example 7: Ultrafine Bubble Concentration-Dependent Biological Threshold Effects

This example demonstrates ultrafine bubble effects show systematic concentration-dependent threshold responses rather than linear dose-response. A 3824-fold concentration increase (1.7×10⁶ to 6.5×10⁹ UFBs/mL) triggers directional reversals across metabolic, immune, and microbial parameters.

To evaluate the relationship between ultrafine bubble concentration and biological outcomes, response patterns between the low-concentration phase (weeks 0-6, 1.7×10⁶ UFBs/mL) and high-concentration phase (weeks 7-12, 6.5×10⁹ UFBs/mL) were compared. This analysis revealed systematic concentration-dependent responses characterized primarily by directional reversals across multiple biological systems (Table 1). Following the 3824-fold concentration increase at week 7, most parameters exhibited complete directional reversals rather than simple dose-response relationships. Butyrate levels shifted from −23.0% (low phase) to +56.2% (high phase), while TNF-α changed from +225.7% to −83.9%, indicating a transition from pro-inflammatory to anti-inflammatory effects. Similarly, valerate, IL-1β, IL-10, and Firmicutes all demonstrated directional reversals between the concentration phases. Bacteroidetes showed enhanced responses in the same direction (+³1.3% to +133.3%, representing a 4.3-fold greater magnitude), while other parameters exhibited complete directional shifts. These systematic reversals occurred across metabolic (SCFAs), immune (cytokines), and microbial (phylum composition) systems, with effects typically detectable by week 8 (2 weeks post-concentration increase). The consistent pattern of directional reversals provides evidence for specific ultrafine bubble biological activity rather than simple dose-response relationships, as such systematic changes across multiple biological systems cannot be attributed to experimental artifacts and indicate coordinated physiological adaptations highly sensitive to concentration thresholds.

TABLE 1
Comparison of biological responses between low and
high UFB concentration phases (test v. control).

| | Low UFB | High UFB | | |
|---|---|---|---|---|
| | Phase* | Phase** | | |
| | Week 6: | Week 12: | | |
| | Test vs. | Test vs. | Magnitude | |
| Parameter | Control | Control | Change | p-Value *** |
| Butyrate change(%) | −23.0 | +56.0 | +79.0 | <0.001 |
| Valerate change(%) | −75.7 | +54.2 | +129.9 | <0.01 |
| TNF-α change (%) | +225.7 | −83.9 | −309.6 | <0.05 |
| IL-1β change (%) | +4.1 | −41.0 | −45.1 | <0.05 |
| IL-10 change (%) | +351.7 | −69.8 | −421.5 | <0.05 |
| Bacteriodetes | +31.3 | +133.3 | +102.0 | >0.05 |
| change(%) | | | | |
| Firmicutes (%) | +56.8 | −56.5 | −113.3 | >0.05 |

*Low UFB Phase: Week 6 test vs. control (1.7 × 10⁶ UFBs/mL).
**High UFB Phase: Week 12 test vs. control (6.5 × 10⁹ UFBs/mL).
*** p-values from independent t-tests comparing test vs. control groups

The above examples and data demonstrate ultrafine bubble water as non-invasive therapeutic intervention for conditions requiring microbiome modulation. Mechanism involves enhanced short chain fatty acid production and reduced inflammatory markers through endogenous microbiome shifts. Natural enhancement through water consumption would be more appealing than short chain fatty acid supplementation. Applications of this technology include gut health products, functional foods, and metabolic health.

### Example 8: Enhancement of Foam Stabilization and Emulsion Stability Via Ultrafine Bubble Technology

Ultrafine bubble-infused water significantly enhances foam stability in oil-water emulsions and beverage systems through interfacial stabilization mechanisms. Ultrafine bubbles position at oil-water interfaces, acting as nano-scale stabilizers that prevent droplet coalescence and maintain foam structure. Effects are sustained over extended time periods (90+ minutes) and demonstrate gas-type dependency in performance characteristics.

Test Formulations—ultrafine bubbles were evaluated across multiple formulation types including oil and water and cosmetic formulations as follows:

Oil and Water Mixing—Canola oil in water recipe: 250 ml water (Hydrosome UFB or DI) and 5 g of food grade canola oil

Cosmetic Formula 1948—Liposome Cream-Gel (oil-in-water emulsion) Recipe: 79 g water (UFB or DI), 6 g Limanthes alba (Meadowfoam) seed oil, 3 g Cocos nucifera (coconut) oil, 3 g sodium acrylate/sodium acryloyldimethyl taurate copolymer/C15-19 alkane/polyglyceryl-6 luarate/polyglycerin-6, 1.5 g Nanochloropis Oculata (micro algae) extract/pullulan, 2 g liposome encapsulated Coenzyme Q10 (ubiquinone) with ascorbyl tetraisopalmitate (Vitamin C) & tocopherol (Vitamin E), 2 g Helcyrhsum stoechas (everlasting) flower extract, 1.5 g Hexylene glycol/caprylyl glycol/wasabia Japonica (wasabi) root extract/Zingiber Officinale (ginger) root extract/Allium sativum (garlic) bulb extract, 300 mg Fragrance

Cosmetic Formula 1031—Conditioning Skin Cream with Botanicals (oil-in-water emulsion) Recipe: 52 g water (UFB or DI), 11.2 Simmondsia chinesis (jojoba) seed oil, 4.8 g mango butter, 2.4 g behentrimonium methosulfate/cetearyl alcohol, 1.6 g Persea gratissima (avocado) oil/glycine

Analytical methods included the following:

Visible Separation: oil slick on liquid surface Visual evaluation of phase separation was conducted at regular intervals following sample preparation. Samples were stored in clear glass vials at room temperature (22±2° C.) and photographed against a standardized white background under consistent lighting conditions. Oil slick formation on the liquid surface was documented at 15-minute intervals for the first 2 hours, then at 30-minute intervals up to 90 minutes total observation time. Digital photographs were taken using a standardized camera setup positioned 15 cm from samples.

Quantitative stability analysis was performed using a LUMiSizer® dispersion analyzer (LUM GmbH, Berlin, Germany) employing analytical photocentrifugation. Samples (0.4 mL) were loaded into rectangular polyamide cells and centrifuged at 3,000 rpm for 2 hours at 25° C. The instrument measured transmission profiles across the sample height every 10 seconds, generating instability index values based on clarification and sedimentation rates. The separation index was calculated as the integral of transmission changes over time, with higher values indicating greater instability. All measurements were performed in triplicate, and results were averaged.

Viscosity measurements were conducted using an Anton Paar ViscoQC L100 rotational viscometer equipped with a motorized Heli-Plus accessory with a T-bar spindle. Samples (50 mL) were equilibrated to room temperature (25° C.) prior to measurement. The spindle was lowered into the sample at the predefined 4 measurement positions. Measurements were taken at 1.5 rpm and data was collected for 60 seconds.

Spreading characteristics were visually assessed by applying 0.1 g samples to the back of the hand in 2 cm diameter circles and photographing spread patterns after 10, 30, and 60 seconds.

Optical microscopy was performed using a compound trinocular microscope (Woehrsh RM). Samples were prepared by placing a small amount between glass slides with standardized compression. Images were captured at 400× magnification under bright-field illumination. For each sample, at least 10 random fields were photographed.

### Results

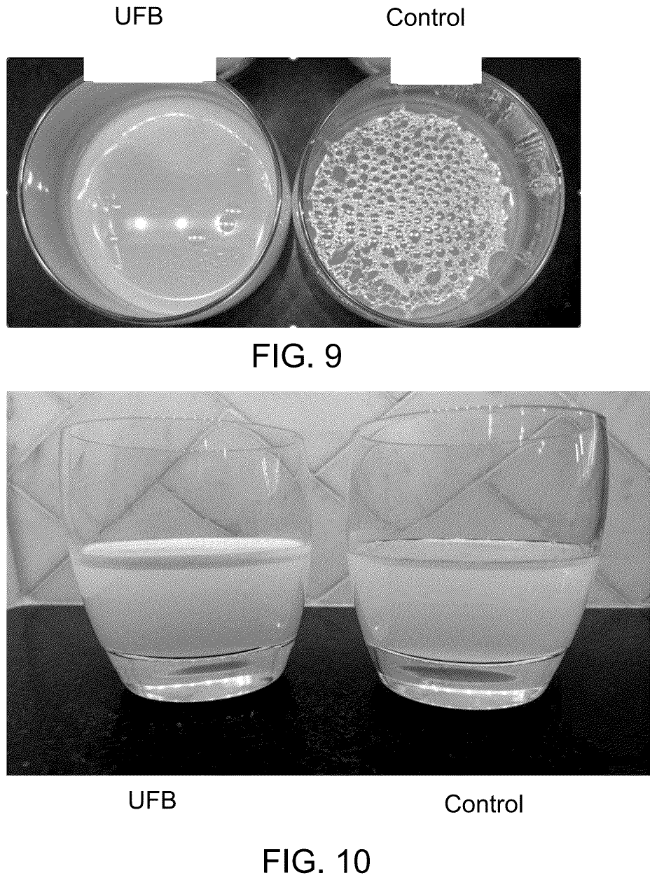

Simple mixing experiments with canola oil demonstrated fundamental differences between UFB-infused water and deionized water controls. UFB formulations maintained stable emulsions throughout the 90-minute observation period, while control samples showed rapid phase separation (FIG. 9). Visual analysis revealed that UFB formulations produced a stable, fine foam layer that persisted throughout the observation period. In contrast, control samples rapidly developed surface oil accumulation with minimal foam formation (FIG. 10).

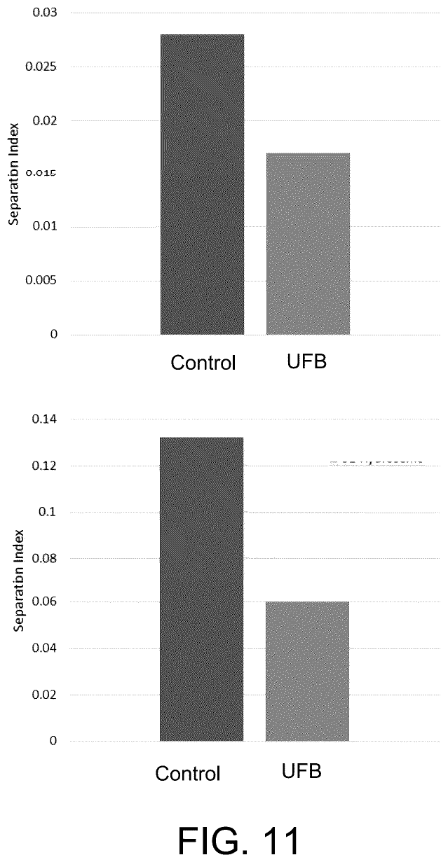

FIG. 11 illustrates the cosmetic emulsion stability Formula 1948 and Formula 01 showed improved stability when formulated with ultrafine bubbles compared to standard deionized water. Lower values indicate better stability.

The ultrafine bubble formulations also showed increased viscosity and microscopic analysis at 400× magnification revealed formulations containing ultrafine bubbles showed small, evenly distributed oil droplets. Ultrafine bubble-formulated cosmetic emulsions exhibited a 39-55% reduction in separation indices compared to control formulations, while oil-water mixing experiments revealed superior resistance to phase separation over 90-minute observation periods.

Microscopic analysis confirmed that UFB formulations produced smaller, more uniformly distributed oil droplets, which correlated with improved sensory properties including faster absorption rates and reduced greasiness. The effectiveness of using ultrafine bubbles without requiring formulation modifications or additional stabilizers provides manufacturers with a streamlined approach to product enhancement while supporting clean-label objectives.

The enhanced stabilization properties of ultrafine bubble technology may enable formulators to reduce conventional emulsifier concentrations while maintaining or improving product performance. This potential reduction in emulsifier usage could lead to significant cost savings and further support clean-label formulation strategies, as fewer synthetic stabilizing agents would be required to achieve desired product characteristics.

### Example 9: Gas-Type Selective Foam Enhancement in Beverage Systems

Gas type selection in ultrafine bubble water enables targeted control of foam characteristics. Nitrogen-filled ultrafine bubbles optimize foam longevity and texture in alcoholic beverages. Air-filled ultrafine bubbles provide enhanced foam volume in sweetener-based systems. This gas-selectivity allows formulation optimization for specific product requirements without altering base ingredients. For example, for beer—improved head retention without additives; nitrogen coffee/cold brew—enhanced creamy texture; protein beverages—stable foam for premium mouthfeel; non-alcoholic beverages requiring foam—clean label enhancement.

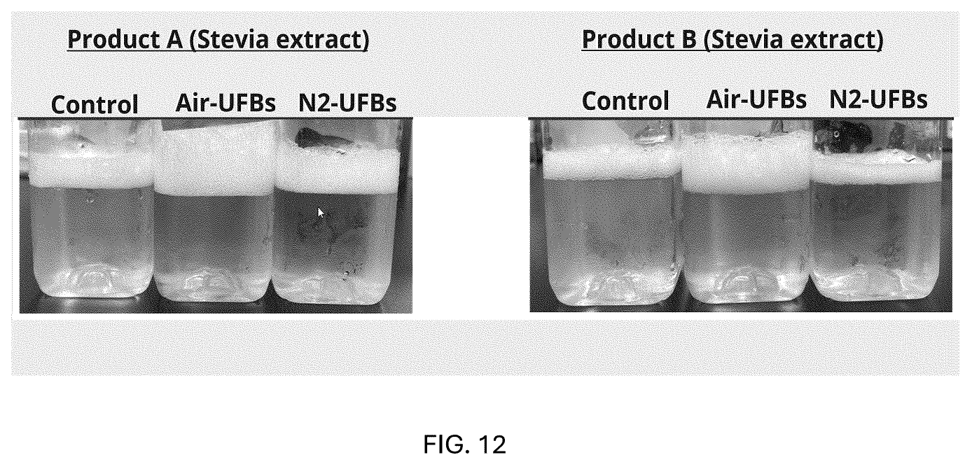

Ultrafine bubbles were used in various beverages applications with different gas compositions according to the present disclosure. Key results in beer applications with nitrogen and ultrafine bubbles (N₂ UFBs) included: enhanced head retention vs. control; improved foam stability over time; Reduced foam collapse rate. Sweetener solutions utilizing ultrafine bubbles included: Air ultrafine bubbles for enhanced foam volume; nitrogen (N₂ UFBs) for similar volume to control but smaller, more uniform bubbles (FIG. 12).

### Example 10: Inhibited Oxidation of Ascorbic Acid (Vitamin C) Facilitated by Ultrafine Bubbles

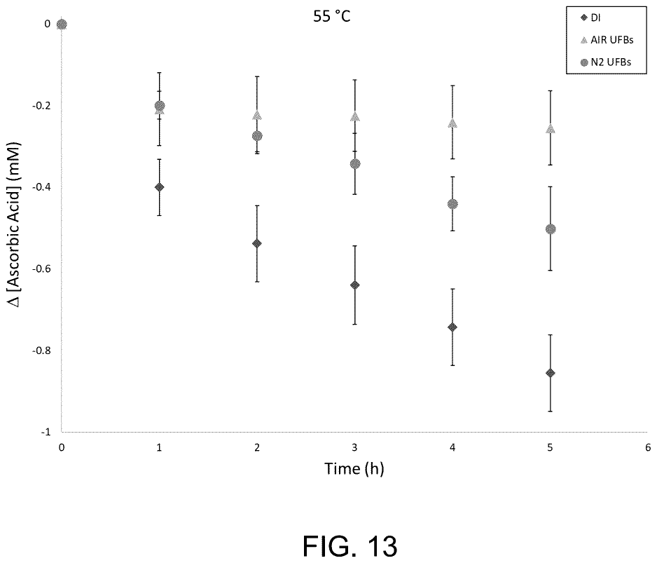

Incorporation of ultrafine bubbles (N2 and Air) to solutions of ascorbic acid as previously described result in the mitigated oxidation of ascorbic acid (Vitamin C) compared to control solutions (deionized water without UFBs). Ultrafine bubbles (UFBs) inhibit the autoxidation of ascorbic acid in aqueous solutions. This inhibitory effect is observed for both Air-UFBs and N₂-UFBs and inhibition does not correlate to type of gas entrained. This is unique from previous reports utilizing H₂-UFBs or specifically utilizing ultra-small UFBs (<10 nm) as antioxidants (FIG. 13). The inhibition of ascorbic acid (Vitamin C) oxidation is advantageous for cosmetic applications, where most formulations opt for more stable, albeit less effect derivatives of Vitamin C. This application could also be extended to beverages or food products that include Vitamin C as an added nutrient to potentially extend their shelf lives.

### Example 11: Ultrafine Bubble Effect on Emulsion Stability and Droplet Size Reduction

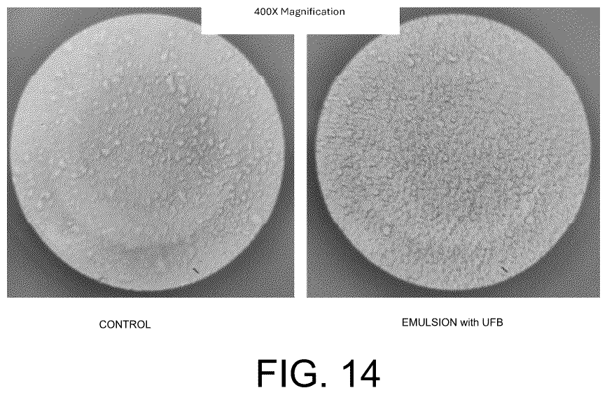

UFBs suspended in an aqueous solution provide added stability when incorporated into emulsified systems (namely O/W emulsions). Emulsion separation indexes were significantly lowered for formulations prepared with UFBs compared to control formulations (deionized water), measured by LUMiSizer™. Visual observation (via microscope) confirmed a decrease in droplet size when UFBs were incorporated into the formulations, as shown in FIG. 14. Specifically, UFBs provide added stability likely due to their reduction in droplet size of the oil dispersed in the water phase.

### Example 12: Enhanced Spray Drying of Oil-In-Water Emulsions Using Ultrafine Bubble Technology

Incorporating ultrafine bubbles into oil-in-water emulsions prior to atomization and drying improves spray drying and product quality. The ultrafine bubble-enriched emulsions demonstrate superior particle size reduction, increased surface area, enhanced stability, and produce spray-dried powders with improved encapsulation efficiency, eliminated surface oil odor, maintained particle integrity, and extended shelf life. An exemplary method for spray drying includes: (a) generating ultrafine bubbles in a liquid carrier, as described herein; (b) combining the UFB-enriched carrier with material to be spray dried to form a feed material; (c) atomizing the feed material; and (d) drying the feed material to form powder particles.

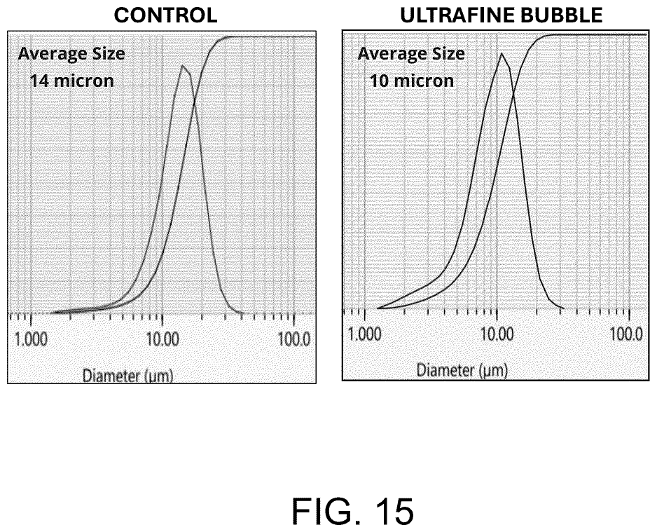

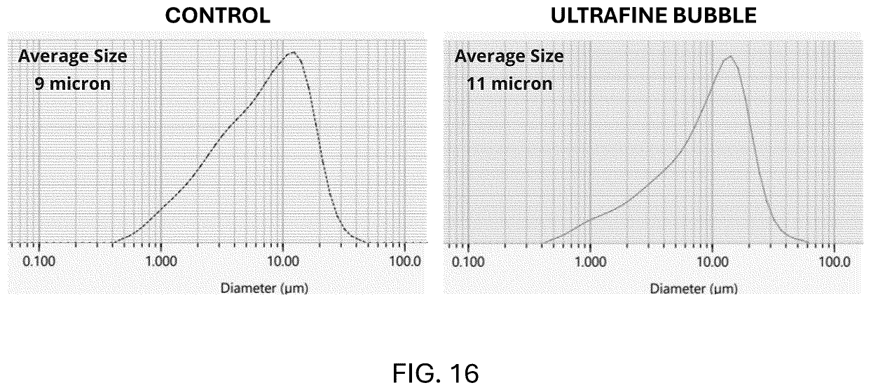

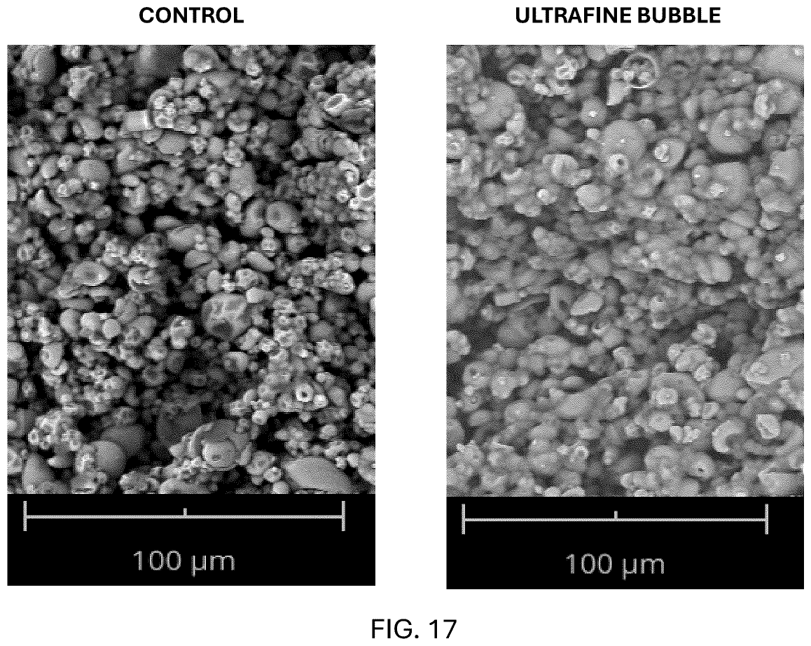

Specifically, in an exemplary embodiment, a modified starch (19% w/w) was mixed with Canola oil (8% w/w) with rotor stator for 30 minutes using ultrafine bubble water or DI water (control). Spray drying trials with 1.5 kg powder production per batch using pilot-scale spray dryer resulted in 29% reduction in emulsion particle size (14 μm control vs. 10 μm UFB; FIG. 12/1), a 48% increase in interfacial surface area (5,155 cm²/cm³ control vs. 7,610 cm²/cm³ UFB), and a 3-fold increase in particle count (8.2×10⁶ particles/mL control vs. 2.4×10⁷ particles/mL UFB) (FIG. 15), prevention of particle collapse during drying (36% size reduction in control vs. 1:1 maintenance in UFB treatment) (FIG. 16), complete elimination of surface oil odor indicating superior encapsulation, spherical particle morphology with smooth, intact surfaces vs. shriveled, irregular control particles (FIG. 17), dry, powdery texture vs. sticky, oily control powder, and enhanced emulsion stability with maintained particle size distribution over time.

### Example 13: Ultrafine Bubble Mediated Viscosity Reduction in Liquid Systems

Incorporation of ultrafine bubbles into liquid systems leads to viscosity reduction compared to control, namely systems incorporated with deionized water. It was found that when UFB are incorporated into liquid systems or during processing of colloidal systems, the viscosity is reduced when compared to controls (i.e., systems without UFBs incorporated).

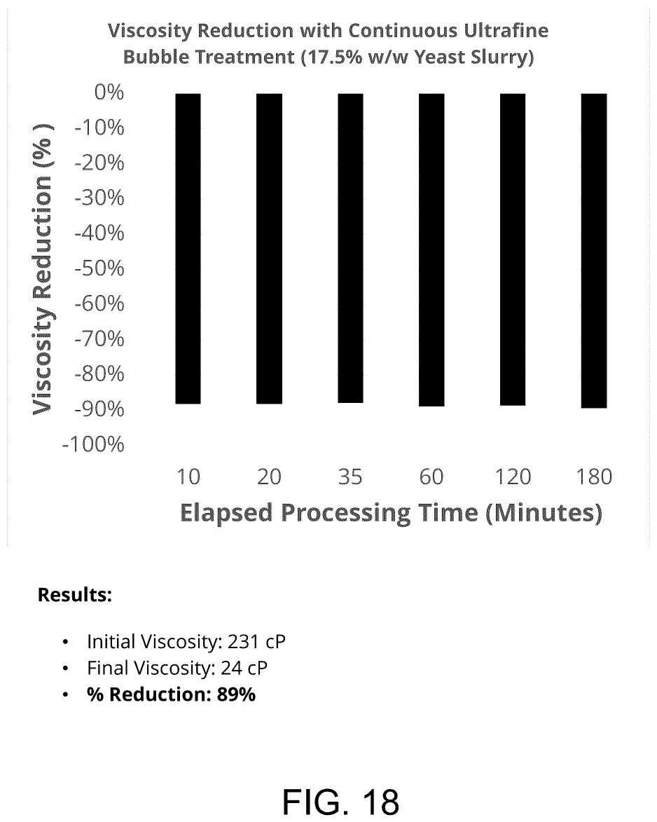

Specifically, 523.56 g of Karo™ light corn syrup was dissolved in 60 g water (either UFB water or DI water) and admixed to form a 17.5% yeast slurry in triplicate. Solutions were allowed to equilibrate at room temperature for 2 hours. Viscosity was measured with Anton Paar ViscoQC L100 with L2 spindle under automatic method and readings taken for 1 minute over time. FIG. 18 illustrates the results of viscosity decrease over time for the 17.5% yeast slurry in the Karo™ syrup/UFB solution. Viscosity for the control (made from DI water) showed a viscosity of 391.83 cP whereas the UFB solution showed a viscosity of 363.23, thereby showing a 7.3% reduction in viscosity in the UFB solution compared to the control solution.

The decrease in viscosity using UFB solutions may be particularly useful in industrial processes where solutions have high viscosity that make it difficult to pump through during the process. By reducing viscosity, UFB solutions are more efficient, having better flow rates and allowing more product to be processed in a smaller time frame, for example.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entireties.

1. A method of modulating gut microbiota composition in a gut of a mammal, the method comprising the steps of:
providing a drinking water composition comprising ultrafine bubbles formed via gaseous cavitation for ingestion by the mammal;
wherein the ultrafine bubbles are present at a concentration in a range of about 1.7×10⁶ to about 1.0×10¹² ultrafine bubbles/mL;
wherein the drinking water composition comprising ultrafine bubbles is ingested by the mammal resulting in modulating microbiota populations within the gut of the mammal.

2. The method of claim 1, wherein the microbiota includes Bacteroidetes populations and Firmicutes populations within the gut of the mammal.

3. The method of claim 2, wherein the method further comprises the step of increasing Bacteroidetes populations within the gut of the mammal and/or a decreasing Firmicutes populations within the gut of the mammal through ingestion of the drinking water composition by the mammal.

4. The method of claim 3, wherein a ratio of Firmicutes/Bacteroidetes within the gut of the mammal is reduced.

5. A method of enhancing short-chain fatty acid production in a gut of a mammal, the method comprising the steps of:
providing drinking water comprising air-filled ultrafine bubbles at a concentration of 1.0×10⁶ to 1.0×10¹² UFBs/mL;
wherein the drinking water composition is ingested by the mammal;
wherein ingestion of the drinking water composition by the mammal increases short-chain fatty acid production in the gut of a mammal.

6. The method of claim 5, wherein ingestion of the drinking water composition by the mammal increases butyrate production in the gut of the mammal by at least 50%.

7. The method of claim 5, wherein ingestion of the drinking water composition by the mammal increases valerate production in the gut of the mammal by at least 60%.

8. The method of claim 5, wherein ingestion of the drinking water composition by the mammal maintains acetate levels in the gut of the mammal.

9. A method of reducing inflammatory markers in a gut of a mammal, the method comprising the steps of:
providing drinking water comprising ultrafine bubbles to the mammal for a period of at least 6 weeks;
wherein ingestion of the drinking water composition by the mammal reduces a level of inflammatory markers in the gut of the mammal, increases short-chain fatty acid production in the gut of a mammal.

10. The method of claim 9, wherein the inflammatory markers include pro-inflammatory (TNF-α, IL-1β) and regulatory (IL-10) cytokines.

11. The method of claim 10, wherein ingestion of the drinking water comprising ultrafine bubbles reduces pro-inflammatory cytokines TNF-α levels by at least 80%.

12. The method of claim 10, wherein ingestion of the drinking water comprising ultrafine bubbles reduces pro-inflammatory cytokines IL-1β levels by at least 40%.

13. The method of claim 10, wherein ingestion of the drinking water comprising ultrafine bubbles reduces regulatory cytokines IL-10 levels by at least 65%.

14. The method of claim 9, wherein reductions in inflammatory markers correlate with changes in gut microbiota composition of the mammal.
